(19) United States
(12) Patent Application Publication
(10) Pub. No.: US 2021/0388383 A1
(43) Pub. Date: Dec. 16, 2021

(54) MODULAR EXPRESSION SYSTEMS FOR GENE EXPRESSION AND METHODS OF USING SAME

(71) Applicant: Children's Hospital Medical Center, Cincinnati, OH (US)

(72) Inventors: Paul R. Andreassen, Cincinnati, OH (US); Helmut Hanenberg, Duesseldorf (DE)

(21) Appl. No.: 17/283,361

(22) PCT Filed: Oct. 11, 2019
(86) PCT No.: PCT/US19/55808

§ 371 Date: Apr. 7, 2021

Related U.S. Application Data
(60) Provisional application No. 62/744,831, filed on Oct. 12, 2018.

(51) Int. Cl. C12N 15/86

(52) U.S. Cl. CPC C12N 15/86; C12N 2740/15043

(57) ABSTRACT

Disclosed herein are compositions and methods for the expression of a gene of interest. The disclosed methods may employ codon-optimization and introduction of non-endogenous restriction sites for efficient expression of a gene. The methods may further employ introduction of a gene variant of interest, such that the disclosed methods, compositions, and systems may be used to determine the significance of a variant of interest. Further disclosed are compositions, systems, and methods for the characterization of gene variants, and other mutations that may impact the function of the protein of interest.

### CROSS-REFERENCE TO RELATED APPLICATIONS

This application claims priority to and benefit of U.S. Provisional Application 62/744,831 filed Oct. 12, 2018. The contents of each are incorporated in their entirety for all purposes.

### STATEMENT REGARDING FEDERALLY-SPONSORED RESEARCH

This invention was made with government support under W81XWH1810269 from the Department of Defense (DOD). The government has certain rights in the invention.

### REFERENCE TO SEQUENCE LISTING SUBMITTED ELECTRONICALLY

The content of the electronically submitted sequence listing (Name: SequenceListing_ST25.txt; Size: 475 bytes; and Date of Creation: Oct. 10, 2019) is incorporated herein by reference in its entirety.

### BACKGROUND

Genetic screens are now being performed for a variety of diseases, ranging from connective tissue diseases and metabolic syndromes to cancer. But the utility of these screens depends on being able to interpret the clinical significance of the variants that are identified. In particular, it is frequently difficult to determine the significance of missense variants, which are often rare. For rare variants, co-segregation studies are frequently underpowered to be useful for variant classification. Unfortunately, there is a basic problem with screening for genetic changes in genes that cause cancer and other diseases when mutated. This problem is that only some mutations cause cancer, while others are harmless. Thus, there is a need to distinguish which mutations have the capacity to cause cancer or other diseases from those which do not.

While functional assays provide the most promising alternative for classifying variants of uncertain significance (VUS), this is oftentimes not available due to the nature of the protein of interest, more particularly, the inability to successfully express a protein for functional assays, especially large proteins. For example, while the gene ATM is known to have ≥2,480 missense VUS listed in ClinVar observed in humans with Ataxia telangiectasia, breast cancer and other cancers, prior to Applicant's invention, there has not been an accurate or high capacity system to functionally classify VUS in this gene. ATM protein expression has been problematic, in part due to large gene size and poor mRNA quality. This has greatly restricted studies to characterize the effects of ATM variants and the roles of different regions of ATM. While ATM has been weakly expressed in human cells using plasmids, a general system to stably and robustly express ATM mutations introduced for the purpose of understanding the function of ATM as a tumor suppressor, cell cycle checkpoint protein, coordinator of the DNA double-strand break (DSB) response, or the study of any variant/mutation of ATM has been lacking. This same deficiency in the art applies to many other genes that are difficult to express either due to their large size, poor mRNA quality, or both. In particular, this is frequently the case for DNA damage response genes.

Another protein that has been problematic to express in mammalian cells is BRCA2. BRCA2, along with BRCA1, is one of the two genes that most frequently cause breast and ovarian cancer when mutated. Biallelic mutation of BRCA2 also causes the D1 subtype of Fanconi anemia (FA), a disease associated with congenital anomalies, progressive bone marrow failure, and a predisposition to leukemia and various types of solid tumors. At the time of the invention, there were approximately 3,400 distinct BRCA2 missense VUS listed in the ClinVar database. Similar to the case for ATM, functional assays provide a greatly needed alternative for characterizing the clinical impact of BRCA2 VUS. Additionally, full-length BRCA2 has not been efficiently and stably expressed at near wild-type levels in mammalian cells using a cDNA, in part because of the very large size of BRCA2 (˜390 kD protein, 10.3 kb cDNA). For BRCA2, as well as for ATM, the full-length protein must generally be expressed for functional assays since there are essential domains at the C-terminus of each protein. The poor quality of the BRCA2 mRNA, which makes it less likely to lead to completion of translation, also complicates expression of BRCA2. In addition to providing a means to functionally characterize VUS, a system for the stable and efficient expression of full-length BRCA2 is needed to better understand the function of BRCA2 as a tumor suppressor. This is important for understanding risks associated with variants/mutants of BRCA2 and also how to therapeutically target tumors that harbor a mutation in BRCA2.

Thus, there is a need in the art for stable and efficient systems for the study of genes that may be larger in size or which may have lower quality mRNA, as defined by suboptimal codon utilization for the particular species, and for which current methods do not allow efficient expression for the study of gene function and/or the effects of mutations. Also, for long genes, PCR-based introduction of mutations can lead to unwanted changes elsewhere due to polymerase errors; it is time-consuming and costly to perform sequencing to ensure that changes are introduced only where desired. Thus, a better alternative for introducing variants/mutations in large cDNAs is needed.

### BRIEF SUMMARY

Disclosed herein are compositions and methods for the expression of a gene of interest. The disclosed methods may employ codon-optimization and introduction of non-endogenous restriction sites for efficient expression of a gene. The methods may further employ introduction of a gene variant of interest, such that the disclosed methods, compositions, and systems may be used to determine the significance of a variant of interest. Further disclosed are compositions, systems, and methods for the characterization of gene variants, and other mutations that may impact the function of the protein of interest.

### BRIEF DESCRIPTION OF THE DRAWINGS

Those of skill in the art will understand that the drawings, described below, are for illustrative purposes only. The drawings are not intended to limit the scope of the present teachings in any way. By way of a brief overview of the following brief description of the drawings, FIG. 2 outlines the modular approach to the generation of expression constructs, while FIGS. 1 and 3-8 present the domain structure of BRCA2, an exemplary expression construct for codon-optimized BRCA2 (BRCA2co), and evidence of expression of BRCA2co in five different cell lines along with correction of defects in the DNA damage response in BRCA2-deficient cells. FIGS. 9-14 display the domain structure of another exemplary protein that may be used with the disclosed methods, ATM, an expression construct for codon-optimized ATM (ATMco), efficient expression of ATMco in two different cell lines along with correction of defects in the DNA double-strand break (DSB) response in ATM-deficient cells. FIG. 15 demonstrates the poor mRNA quality of six candidates for expression using our modular codon-optimized approach (BRCA1, BRCA2, ATM, ATR, CHEK2 and FANCM), many of which are difficult to express due to their large size and/or the poor mRNA quality. FIGS. 16-17 demonstrate unique restriction sites, all generated during creation of the codon-optimized cDNA, in BRCA2co and ATMco that can be utilized for rapid and error-free insertion of synthetic fragments that contain variants or mutations. The figures are intended to be exemplary in nature, and not intended to be limiting in any way to the disclosed compositions and methods.

FIG. 1. Diagram of key domains and interacting regions of BRCA2. The function of much of the protein is unknown, in part due to difficulties expressing the full-length protein, which limits functional studies.

FIG. 2. Schematic of novel system for the streamlined (and rapid) generation of codon-optimized cDNAs for genes containing VUS and other variants or mutations. DNA fragments that contain variants are synthesized commercially in batches, error-free. Fragments that correspond to unique restriction sites are inserted into a vector, in this case the p2CL lentiviral backbone that contains the codon-optimized gene. Inserted fragments and junctions are sequenced to ensure accuracy. For viral vectors, virus is packaged and either used directly to infect target cells to express the gene of interest or frozen for future use.

FIG. 3. Diagram of codon-optimized BRCA2 (BRCA2co) in the p2CL lentiviral backbone. The total size of the lentiviral vector as diagramed is 15,161 bp (with BRCA2co being 10,254 bp itself without a N-terminal Flag-HA epitope tag that can be added).

FIG. 4. Efficient expression of human BRCA2 in multiple different cell lines including genetically-deficient human FA-D1 cells and a BRCA2-deficient ovarian cancer line. (A) Codon-optimized (“co”) BRCA2 is stably expressed in EBV-transformed lymphoblasts (LCLs) from a FA-D1 patient at levels similar to those in a non-FA control line. (B) Stable expression of BRCA2co in primary FA-D1 fibroblasts. (C) WT or mutant BRCA2 is detected in transduced PE01 ovarian cancer cells. BRCA2 protein is not expressed in cells that contain the vector alone (A-C). Actin is shown as a loading control.

FIG. 5A-5D. Functional correction of human FA patient-derived cell lines with a genetic-deficiency for BRCA2. While full-length human BRCA2 has not previously been stably expressed in human cells using a cDNA, such expression here enables tests that distinguish the effects of benign and pathogenic variants of BRCA2. (FIG. 5A) Survival of LCLs from a FA-D1 patient containing the control vector or different forms of BRCA2co following treatment with MMC, as compared to non-FA LCLs with endogenous WT BRCA2. The c.3G>A BRCA2 variant (mut-BRCA2co) was included. WT BRCA2co protein, either with or without a N-terminal Flag-HA tag, fully restored resistance to MMC. (5B) Relative survival of FA-D1 LCLs following treatment with a PARP inhibitor (olaparib). The same labels for cells with different forms of BRCA2co apply to both (FIGS. 5A and 5B). (FIG. 5C) Quantification of RAD51 foci formation in FA-D1 fibroblasts reconstituted with different forms of BRCA2co, either before or 16 hr after exposure to 10 Gy IR. (FIG. 5D) The relative ability of WT BRCA2 (100%) or variants to correct defective HR in cells shRNA-depleted of BRCA2 (0% in cells containing vector; Vec), as measured by flow cytometry in U20S-DR cells with a reporter construct, as described in Zhang F, Fan Q, Ren K, Andreassen P R. PALB2 functionally connects the breast cancer susceptibility proteins BRCA1 and BRCA2. Mol Cancer Res. 2009; 7(7):1110-1118.). Differences between WT/benign variants and Vec/pathogenic variants are significant (P<0.05).

FIG. 6. Partial correction of IR sensitivity by the p.F590C variant of BRCA2 in FA-D1 fibroblasts, as evidence of the functional importance of the interaction of BRCA2 with another tumor suppressor, RAD51C. Resistance to IR for BRCA2-deficient FA-D1 fibroblasts transformed by SV40 Lg. T and reconstituted with different forms of full-length BRCA2 was measured using colony formation assays. Results were normalized to untreated cells for each form of BRCA2. Differences between cells corrected with WT or p.C554W, and p.F590C BRCA2, are significant (p<0.005).

FIGS. 7A and 7B. RAD51C directly binds to an uncharacterized region of the BRCA2 protein and this interaction between the products of two breast/ovarian cancer tumor suppressor genes is disrupted by breast-ovarian cancer-associated variants. (7A) The 540-600 amino acid region of BRCA2, fused to the N-terminus of GFP, immunoprecipitates RAD51C but not RAD51, when expressed in 293T cells. (7B) The F590C variant, introduced into the 1-969 fragment of BRCA2 along with a N-terminal Flag-HA tag, disrupts interaction with RAD51C when expressed in 293T cells, as determined using an immunoprecipitation assay performed with anti-Flag beads. The indicated proteins were then detected by immunoblotting.

FIG. 8. Stable shRNA-resistant expression of BRCA2co in immortalized MCF10A cells, which are non-transformed human mammary epithelial cells. Endogenous BRCA2 was depleted from MCF10A cells using a shRNA (shB2) against the 5′-GAAGAATGCAGGTTTAATA (SEQ ID NO: 1) target sequence (left). Flag-HA tagged BRCA2co was efficiently expressed in these cells and is shRNA-resistant, as shown by immunoblots with anti-HA antibodies (right). Actin is shown as a loading control.

FIG. 9. Known domains of human ATM. There are seven defined domains: a substrate-binding domain (amino acids 91-97); a nuclear localization signal (NLS, amino acids 385-388); a leucine zipper motif (amino acids 1217-1239), and four domains at the C-terminus (Fatkin) that have a role in ATM kinase activity and which are conserved in phosphatidyl-3 kinase-related kinases (PIKKS): FAT (amino acids 1966-2566), kinase (catalytic) domain (amino acids 2712-2960), PIKK regulatory Domain (PRD, amino acids 2961-3025) and FATC (amino acids 3026-3056). There are 49 HEAT repeats (generally 30-55 amino acids in length) distributed from amino acids 1-2652 which are not shown for the sake of simplicity. Additionally, a TAN (amino acids 15-27) and a NBS1-binding region identified in the yeast ATM homolog are not shown here because they have either not been functionally tested or confirmed in mammalian cells. Much of ATM has unknown function, in part due to limited studies due to difficulties expressing the full-length protein.

FIG. 10. Codon-optimized ATM (ATMco) in the p2CL lentiviral backbone. ATMco is 9,168 bp; the overall construct is 14,075 bp. The vector contains IRES-neomycin to ensure that all G418-selected cells express ATMco.

FIG. 11A-11B. Robust expression of human ATM in two different genetically-deficient cells from different A-T patients. 11A) Expression of full-length ATMco in AT1-T and AT2-T fibroblasts. All cells were immortalized with SV40 Lg T antigen (T). Controls: A-T cells transduced with the empty vector (“+Vec”) lack detectable endogenous ATM; GM00038C-T cells (non A-T) display normal levels of endogenous ATM. B) AT2-T A-T fibroblasts were transduced with different versions of ATMco [wild-type (WT), 2 benign, and 2 pathogenic variants. ATM protein is detected using GeneTex antibody (2C1) (A-B) and HA-antibody recognizing the Flag-HA epitope tag (11B); actin, loading control.

FIG. 12A-12D. ATMco corrects defects in multiple aspects of DNA damage signaling in ATM-deficient cells. Thus, while full-length ATM has not been previously expressed in human cells using a cDNA, the expression system utilized here allows tests of ATM function that distinguish the effects of benign and pathogenic variants of ATM. 12A) Western blot of pS1981-ATM and pT68-CHK2 in AT1-T ATM-deficient fibroblasts transduced with empty vector, WT ATMco, or 1 of 4 variants of ATMco. Variants: 12030C and L2332P, benign; R2227C and V2424G, pathogenic. Levels of non-phospho-specific CHK2 did not vary with the form of ATMco expressed; actin, loading control. 12B) Example of pT68-CHK2 foci in ATM-deficient AT2-T fibroblasts from an A-T patient transduced with empty vector or WT ATMco. 12C) The percentage of AT2-T cells transduced with empty vector or different forms of ATMco, with 5 or more pCHK2 foci. Cells were processed as previously described. (Zhang F, Fan Q, Ren K, Andreassen P R. PALB2 functionally connects the breast cancer susceptibility proteins BRCA1 and BRCA2. Mol Cancer Res. 2009; 7(7):1110-1118.) Differences between cells corrected with WT-ATM or benign variants, versus cells containing the empty vector or pathogenic variants, were significant (p<0.001). 12D) To detect acetylation of ATM, cell lysates from ATM-deficient AT1-T cells transduced with empty vector, as a negative control, or with WT ATMco, were Western blotted (left) or immunoprecipitated using anti-Flag antibodies and then Western blotted (right) using an anti-acetyl antibody as described previously. (Sun Y, Xu Y, Roy K, Price B D. DNA damage-induced acetylation of lysine 3016 of ATM activates ATM kinase activity. Mol Cell Biol. 2007; 27(24):8502-8509.) ATMco contained a Flag-HA epitope tag. In A-D, cells were collected or fixed 45 minutes after exposure to 5 Gy IR.

FIG. 13A-13C. ATMco and benign, but not pathogenic, variants stably expressed in ATM-deficient cells are functional for cellular resistance and G2 checkpoint arrest in response to IR. This demonstrates the ability of ATMco to distinguish the effects of benign and pathogenic variants. 13A) AT2-T fibroblasts were stably transduced with WT ATM (“ATMco” and “FH-ATMco”), or 1 of 4 variants of ATMco. Controls included A-T fibroblasts that were transduced with empty vector “+Vec” and “non A-T” fibroblasts. Cells were then treated with a range of doses of IR and assayed for relative survival. The R2227C and V2424G pathogenic variants had a slight residual activity as compared to cells reconstituted with empty vector. 13B-13C) AT2-T A-T fibroblasts were stably transduced with empty vector “+Vec” or different forms of ATMco as in A and were analyzed for G2 checkpoint function utilizing flow cytometry with phospho-histone H3 (pH3), as described. (Andreassen P R, Skoufias D A, Margolis R L. Analysis of the spindle-assembly checkpoint in HeLa cells. Methods Mol Biol. 2004; 281:213-225 and Taniguchi T, Garcia-Higuera I, Xu B, et al. Convergence of the Fanconi anemia and ataxia telangiectasia signaling pathways. Cell. 2002; 109(4):459-472.) Cells were fixed for analysis at 2.5 hr after exposure to 5 Gy IR. 13B) Representative dot plots with mitotic cells (pH3+ cells with a 4C DNA content measured using propidium iodide staining) indicated by boxes. 13C) The percentage of cells in mitosis following exposure to IR is displayed relative to untreated populations for cells with each genotype. A strong reduction in mitosis is indicative of a functional G2 checkpoint. A & C were performed in triplicate and the mean is shown for each data point. Differences for A-T cells corrected with WT ATM-co, and 12030V and S1983N, as compared to other forms of ATM were significant (p<0.001) in 13A & 13C.

FIG. 14A-14C. Functional assays of internal deletion mutants expressed in ATM-deficient cells display the ability of ATMco to functionally test the role of mutants defective for distinct domains in ATM. Therefore, these expression and assay systems can be utilized to characterize the roles of domains and specific residues throughout ATM. ATM-deficient AT2-T A-T fibroblasts transduced with empty vector (“+Vec”), WT ATMco, or 1 of 3 internal deletion (loopout) mutations of ATMco. Mutations: AR90-N143; AF1287-H1516); AP2353-P2553. 14A) Western blots of the indicated phosphoproteins. Levels of different forms of FH-ATMco are indicated using anti-HA antibodies. Actin, loading control. 14B) Assays of the assembly of pCHK2 foci (>5 foci/cell) 45 min after treatment with 5 Gy IR. The index is the same for B & C. 14C) G2 checkpoint assays 2.5 hr after exposure to 5 Gy IR, performed using flow cytometry by detecting and quantifying mitotic cells with pH3 antibodies. The % mitotic cells are shown for each cell line relative to levels in untreated populations. Differences between cells reconstituted with FH-ATMco-WT versus all other forms of ATMco and the vector control are significant in B (p<0.001) and C (p<0.01).

FIG. 15A-15G. Plots of RNA quality across multiple genes. (A-G) The mRNA quality is poor throughout BRCA1, BRCA2, ATM, ATR, CHEK2, FANCM, and PRKDC, many of which are very long genes. These are strong candidates for expression using the invention disclosed herein. For each gene, the frequency of codons with increasingly poor quality, based in part on sub-optimal codon utilization for particular amino acids in humans, is shown to the left, while low quality codons are seen throughout the mRNA (3′-5′) for many of these genes, such as ATM, as shown to the right. Plots were generated utilizing software available at https://www.thermofisher.com/us/en/home/life-science/cloning/gene-synthesis.html.

FIG. 16. Multiple potential unique cloning sites are present throughout codon-optimized BRCA2. None of these restriction sites are endogenous, meaning they were not present at the corresponding position in the wild-type BRCA2 gene. Unique restriction sites generated by codon-optimization due to accompanying alterations in the sequence, or investigator-introduced silent restriction sites, are highlighted and their positions indicated relative to the start site for transcription.

FIG. 17. Multiple potential unique cloning sites are present throughout codon-optimized ATM. None of these restriction sites are endogenous, meaning they were not present at the corresponding position in the wild-type ATM gene. As such, all unique restriction sites were generated by codon-optimization due to accompanying alterations in the sequence, or were investigator-introduced silent restriction sites, and are highlighted and their positions indicated relative to the start site for transcription.

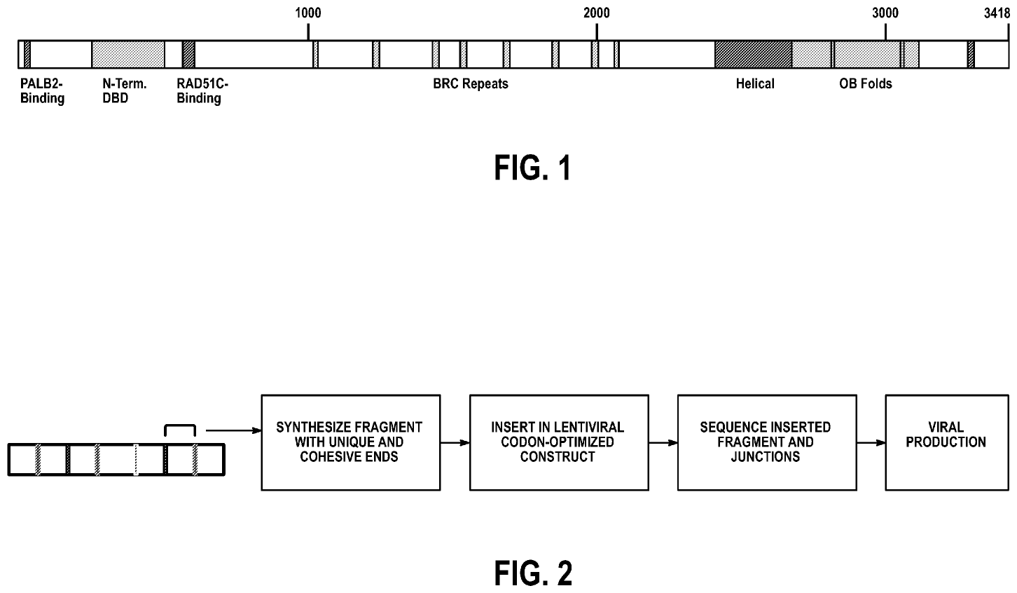

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

“Sequence identity” as used herein indicates a nucleic acid sequence that has the same nucleic acid sequence as a reference sequence, or has a specified percentage of nucleotides that are the same at the corresponding location within a reference sequence when the two sequences are optimally aligned. For example a nucleic acid sequence may have at least 50%, 60%, 70%, 75%, 80%, 85%, 90%, 95%, 96%, 97%, 98%, 99%, or 100% identity to the reference nucleic acid sequence. The length of comparison sequences will generally be at least 5 contiguous nucleotides, preferably at least 10, 11, 12, 13, 14, 15, 16, 17, 18, 19, 20, 21, 22, 23, 24, or 25 contiguous nucleotides, and most preferably the full length nucleotide sequence. Sequence identity may be measured using sequence analysis software on the default setting (e.g., Sequence Analysis Software Package of the Genetics Computer Group, University of Wisconsin Biotechnology Center, 1710 University Avenue, Madison, Wis. 53705). Such software may match similar sequences by assigning degrees of homology to various substitutions, deletions, and other modifications.

Genetic screening is now recommended for all women diagnosed with ovarian cancer. The detection of mutations in cancer genes can be used to guide both cancer prevention and treatment in patients with ovarian, breast and other cancers. However, at present, information from these screens is underutilized since the clinical significance of many of the variants that are identified is unclear, and there is a need for a more complete understanding of variants that are identified, but are of unknown significance. The instant disclosure addresses this deficiency in the art in two important ways. First, BRCA2 is one of the most frequently mutated genes that can cause ovarian or breast cancer. Due in part to difficulties expressing full-length BRCA2 and its variants, functional assays that could aid in interpreting the significance of BRCA2 variants have been of limited utility. To empower the life-saving potential of genetic screens for breast and ovarian cancer, Applicant has developed a method for efficiently expressing codon-optimized BRCA2 for rapid functional assays of BRCA2 variants. Notably, Applicant has demonstrated that the disclosed methods can distinguish full, partial or no loss of function associated with disease-related variants of BRCA2.

Previous systems were often based on knock-in of an alteration to the mouse BRCA2 gene, for example, by a process called recombineering. By this process, homologous recombination is carried out at random double-strand breaks proximal to the desired locus (at a low frequency) using a donor template with the desired sequence alteration and homologous arms in mouse embryonic stem (ES) cells. The instant disclosure provides a superior, more rapid and more efficient approach to expression of difficult-to-express genes. Prior art methods such as the recombineering method, as described above, are labor intensive and time consuming, requiring the steps of selecting, growing, and confirming cells with the desired change, and occurs at a very low frequency. In contrast, the disclosed methods are comparatively rapid. The methods employ a codon-optimized (“co”) gene with engineered restriction sites for introduction of variants that may be synthesized in batches. As a result, for the first time, efficient and stable expression of larger genes, and genes which have been difficult to express—such as BRCA2 or ATM and many others—can be achieved.

In one aspect, the methods may be used for identifying deleterious and/or pathogenic missense variants of genes that previously have not be able to be expressed due to various factors (RNA stability and/or size of the gene, as mentioned above). The methods may further allow for defining the function of domains throughout such genes or the function of other residues in the encoded protein, at a rate that was previously not possible due to inefficient methods or in certain cases, a complete inability to express a gene of interest. In one aspect, the methods may be used to empower genetic screens by providing a basis for the systematic interpretation of VUS, including missense alterations and small insertions/deletions and may allow for identification of individuals harboring pathogenic variants that would benefit from increased surveillance and preventative treatments which are potentially lifesaving.

The disclosed methods can be used for the expression and characterization of genes which previous to Applicant's invention, could not be efficiently expressed. While exemplary genes include breast and ovarian cancer genes, and/or genes associated with the DNA damage response, any gene that is large and/or which has poor mRNA quality which makes them difficult to express may be used with the disclosed invention.

In one aspect, the methods disclose a method for expressing a gene of interest. The method may employ the use of an expression vector comprising a codon-optimized cDNA of the gene of interest. By “codon-optimization” it is meant the substitution of a codon with a low frequency of utilization to that of a codon with a higher frequency of utilization for a particular species. Codon utilization is similar in vertebrates such as humans and mice, but this can differ greatly from which specific codon is preferred for a particular amino acid in “lower organisms” such as E. Coli, Yeast or Maize. For many human genes, such as BRCA1, BRCA2, ATM, ATR, CHEK2, FANCM and DNA-PKcs, which are typically very large, a poor quality mRNA that cannot be efficiently translated results from the utilization of codons across the gene that may be utilized at a higher frequency in lower organisms than in humans and other mammals. As such, codon-optimization typically includes switching from codons with a lower frequency of utilization in humans to one with a higher frequency of utilization across the entire cDNA. Typically, codons are optimized across the genome—and while there may be two codons that are utilized with a similar frequency—for example, 38% and 36% (with others being used at a lower frequency)—the highest will generally be used. It will be understood by one of ordinary skill in the art that such substitution/optimization with a specific “most frequent” codon will not always be the case due to placement of a customized restriction site or to obtain a more balanced/desirable GC content, and that in certain instances a codon of lesser frequency may be used, while still codon-optimizing the gene within the scope of the instant disclosure. In other words, it will be understood by one of ordinary skill in the art that the codon-optimization may not be 100% codon-optimized throughout due to the introduction of customized restriction sites. Selection of codons may be guided by an algorithm, for example, that available at https:www.thermofisher.com/us/en/home/life-scien/cloning/gene-synthesis/geneart-gene-synthesis/geneoptomizer.html!SID=fr-geneart-5.

Table 1. The following table demonstrates that different species often prefer utilization of different codons. It is generally assumed that for codon-optimization of a human (or mammalian gene), if a low frequency codon is present in the natural gene, expression can be improved by changing such codons to the most frequently utilized (presumably optimal) codon for the particular species. Codon-optimization (synthesizing a cDNA with each or nearly all (at least about 80%, at least about 85%, at least about 90%, or at least about 95%) codon(s) representing the most frequently utilized for that species) can improve the efficiency of translation. This is likely a significant issue for long genes such as ATM and BRCA2, where translation may never be completed due to codons that are not efficiently utilized by the translational machinery in that species.

| | | Fraction of specific codon utilization for | | | | |
|---|---|---|---|---|---|---|
| | | each particular amino acid by species | | | | |
| Amino | | E. Coli | | | | |
| Acid | Codon | (Bacteria) | Yeast | Human | Mouse | Maize |
| A | GCT | 0.18 | 0.38 | 0.26 | 0.29 | 0.25 |
| A | GCC | 0.26 | 0.22 | 0.40 | 0.38 | 0.33 |
| A | GCA | 0.23 | 0.29 | 0.23 | 0.23 | 0.19 |
| A | GCG | 0.33 | 0.11 | 0.11 | 0.10 | 0.23 |
| C | TGT | 0.46 | 0.63 | 0.45 | 0.48 | 0.34 |
| C | TGC | 0.54 | 0.37 | 0.55 | 0.52 | 0.66 |
| D | GAT | 0.63 | 0.65 | 0.46 | 0.44 | 0.44 |
| D | GAC | 0.37 | 0.35 | 0.54 | 0.56 | 0.56 |
| E | GAA | 0.68 | 0.71 | 0.42 | 0.40 | 0.36 |
| E | GAG | 0.32 | 0.29 | 0.58 | 0.60 | 0.64 |
| F | TTT | 0.58 | 0.59 | 0.45 | 0.43 | 0.37 |
| F | TTC | 0.42 | 0.41 | 0.55 | 0.57 | 0.63 |
| G | GGT | 0.35 | 0.47 | 0.16 | 0.18 | 0.21 |
| G | GGC | 0.37 | 0.19 | 0.34 | 0.33 | 0.39 |
| G | GGA | 0.13 | 0.22 | 0.25 | 0.26 | 0.20 |
| G | GGG | 0.15 | 0.12 | 0.25 | 0.23 | 0.21 |
| H | CAT | 0.57 | 0.64 | 0.41 | 0.40 | 0.43 |
| H | CAC | 0.43 | 0.36 | 0.59 | 0.60 | 0.57 |
| I | ATT | 0.49 | 0.46 | 0.36 | 0.34 | 0.33 |
| I | ATC | 0.39 | 0.26 | 0.48 | 0.50 | 0.47 |
| I | ATA | 0.11 | 0.27 | 0.16 | 0.16 | 0.20 |
| K | AAA | 0.74 | 0.58 | 0.42 | 0.39 | 0.30 |
| K | AAG | 0.26 | 0.42 | 0.58 | 0.61 | 0.70 |
| L | CTT | 0.12 | 0.13 | 0.13 | 0.13 | 0.18 |
| L | CTC | 0.10 | 0.06 | 0.20 | 0.20 | 0.25 |
| L | CTA | 0.04 | 0.14 | 0.07 | 0.08 | 0.08 |
| L | CTG | 0.47 | 0.11 | 0.41 | 0.39 | 0.25 |
| L | TTA | 0.14 | 0.28 | 0.07 | 0.06 | 0.08 |
| L | TTG | 0.13 | 0.29 | 0.13 | 0.13 | 0.15 |
| M | ATG | 1.00 | 1.00 | 1.00 | 1.00 | 1.00 |
| N | AAT | 0.49 | 0.59 | 0.46 | 0.43 | 0.40 |
| N | AAC | 0.51 | 0.41 | 0.54 | 0.57 | 0.60 |
| P | CCT | 0.18 | 0.31 | 0.28 | 0.30 | 0.24 |
| P | CCC | 0.13 | 0.15 | 0.33 | 0.31 | 0.24 |
| P | CCA | 0.20 | 0.41 | 0.27 | 0.28 | 0.26 |
| P | CCG | 0.49 | 0.12 | 0.11 | 0.10 | 0.26 |
| Q | CAA | 0.34 | 0.69 | 0.25 | 0.25 | 0.39 |
| Q | CAG | 0.66 | 0.31 | 0.75 | 0.75 | 0.61 |
| R | AGA | 0.07 | 0.48 | 0.20 | 0.21 | 0.16 |
| R | AGG | 0.04 | 0.21 | 0.20 | 0.22 | 0.25 |
| R | CGT | 0.36 | 0.15 | 0.08 | 0.09 | 0.12 |
| R | CGC | 0.36 | 0.06 | 0.19 | 0.18 | 0.23 |
| R | CGA | 0.07 | 0.07 | 0.11 | 0.12 | 0.09 |
| R | CGG | 0.11 | 0.04 | 0.21 | 0.19 | 0.15 |
| S | AGT | 0.16 | 0.16 | 0.15 | 0.15 | 0.11 |
| S | AGC | 0.25 | 0.11 | 0.24 | 0.24 | 0.21 |
| S | TCT | 0.17 | 0.26 | 0.18 | 0.19 | 0.17 |
| S | TCC | 0.15 | 0.16 | 0.22 | 0.22 | 0.22 |
| S | TCA | 0.14 | 0.21 | 0.15 | 0.14 | 0.15 |
| S | TCG | 0.14 | 0.10 | 0.06 | 0.05 | 0.14 |
| T | ACT | 0.19 | 0.35 | 0.24 | 0.25 | 0.24 |
| T | ACC | 0.40 | 0.22 | 0.36 | 0.35 | 0.33 |
| T | ACA | 0.17 | 0.30 | 0.28 | 0.29 | 0.22 |
| T | ACG | 0.25 | 0.13 | 0.12 | 0.11 | 0.21 |
| V | GTT | 0.28 | 0.39 | 0.18 | 0.17 | 0.24 |
| V | GTC | 0.20 | 0.21 | 0.24 | 0.25 | 0.29 |
| V | GTA | 0.17 | 0.21 | 0.11 | 0.12 | 0.11 |
| V | GTG | 0.35 | 0.19 | 0.47 | 0.46 | 0.36 |
| W | TGG | 1.00 | 1.00 | 1.00 | 1.00 | 1.00 |
| Y | TAT | 0.59 | 0.56 | 0.43 | 0.43 | 0.37 |
| Y | TAC | 0.41 | 0.44 | 0.57 | 0.58 | 0.63 |
| Ter | TAA | 0.61 | 0.48 | 0.28 | 0.26 | 0.24 |
| Ter | TAG | 0.09 | 0.24 | 0.20 | 0.22 | 0.32 |
| Ter | TGG | 0.30 | 0.29 | 0.52 | 0.52 | 0.44 |

The codon-optimized gene may further comprise at least two non-endogenous restriction sites, wherein the at least two non-endogenous restriction sites are present at an interval of not more than 2000 base pairs, or not more than 1500 base pairs, or at an interval of between about 100 base pairs to about 1500 base pairs, or an interval of between 250 base pairs to 1000 base pairs, or about 500 base pairs to about 750 base pairs. In certain aspects, there may be at least 3, or at least 4 or at least 5, or at least 6, or at least 7, or at least 8, or at least 9, or at least 10, or at least 11, or at least 12, or at least 13, or at least 14, or at least 15, or at least 16, or at least 17, or at least 18, or at least 19, or at least 20 restriction sites introduced into the full length cDNA for a gene. The non-endogenous restriction site may be, in one aspect, unique to the gene of interest. By “unique,” it is meant that the restriction site occurs only once in the particular codon-optimized cDNA. Pairs of these unique restriction sites can be utilized to introduce synthesized fragments, with or without a variant in the codon-optimized cDNA. The non-endogenous and/or unique restrictions sites may be introduced during the codon-optimization process, for example, wherein the codon-optimization causes a restriction site to be introduced into the sequence.

In one aspect, the gene may be characterized as having an undesirable expression efficiency and/or poor mRNA quality. For example, the gene may have a length such that gene expression efficiency is reduced or compromised using methods known in the art. In one aspect, the gene used in the disclosed methods may have a length of greater than about 5 kb, or about 6 kb, or about 7 kb, or about 8 kb. At 6 kb, for example, it is generally accepted that viral vectors typically do not yield significant expression, driven by a roughly log drop-off in expression for each additional 2 kb of insert in a typical vector.

In one aspect, the gene may be selected from one of the following non-limiting list of genes: ataxia telangiectasia mutated serine/threonine protein kinase (ATM);); ataxia telangiectasia and Rad3-related protein kinase (ATR); breast cancer 1, early onset (BRCA1); breast cancer 2, early onset (BRCA2); checkpoint kinase 1 (CHEK1); Fanconi anemia complementation group M (FANCM); and protein kinase, DNA-activated, catalytic subunit (PRKDC or “DNA-PKcs”). BRCA1 and CHEK1 are known to have poor mRNA quality and have been difficult to express. BRCA2, FANCM and PRKDC are all greater in length than the typical 6.0 kb cutoff at which detectable protein is often not detected using lentiviral vectors. Accession numbers will be readily appreciated by one of skill in the art but are provided herein for convenience and for the sake of clarity: hATM: ACCESSION NM_000051 9.0 kb; ATR ACCESSION NM_001184-variant 1. 7935 bp, 2645 bp; hBRCA1 ACCESSION NM_007294-variant 1. 5592 bp, 1864 amino acids; hBRCA2: ACCESSION NM_000059 10.3 kb; CHEK2 ACCESSION NM_007194 1632 bp, 544 amino acids; PRKDC (DNAPKcs) ACCESSION NM_006904-variant 1, (longer variant) 12387 bp, 4129 amino acids; FANCM ACCESSION NM_020937 6147 bp, 2645 amino acids.

In one aspect, the method may further comprise synthesizing a fragment of the codon-optimized cDNA using methods well known to one of ordinary skill in the art. The fragment may then be inserted into an expression vector. Construction of a gene fragment can be accomplished using any suitable genetic engineering technique, such as those described in Ausubel et al. (Current Protocols in Molecular Biology, John Wiley & Sons, New York, 2000). Many techniques of transgene construction and of expression constructs for transfection or transformation in general are known and may be used to generate the desired sequences. The fragment may be of any size deemed acceptable by one of ordinary skill in the art for use in the disclosed methods, and may be, for example from about 10 base pairs to about 3000 base pairs, or from about 50 base pairs to about 2500 base pairs, or from about 100 base pairs to about 2000 base pairs, or from about 200 base pairs to about 1500 base pairs, or from about 500 base pairs to about 1000 base pairs.

The method may further comprise the step of synthesizing a fragment of the codon-optimized cDNA as described above, wherein the fragment is inserted into an expression vector, and wherein the fragment of the codon-optimized cDNA comprises a variant of said cDNA. As used herein, the term “variant” is intended to encompass that definition as used by one of ordinary skill in the art in the field of molecular biology or genetics, in particular, including any mutation in the sequence as compared to wild-type sequence, in particular, a mutation of interest. The variant may be, in particular, a variation in the gene that occurs naturally in the population or which is inherited or occurs somatically, resulting in a mutation that is suspected of contributing to function, or malfunction of the gene. Such variant may be tested using the expression system described herein, followed by any method known to test the function of the resulting gene product. In one aspect, the gene or gene fragment may comprise a variant that is a mutation selected from one or more of a missense mutation, a nonsense mutation, an insertion, a deletion, a duplication, a frameshift mutation, a repeat expansion mutation, that occurs in individuals or which is an artificial mutation introduced to test protein function, wherein said mutation is intentionally introduced into said gene. The terms “variant” and “mutation” may be used interchangeably herein unless a distinction is made.

Using the described methods, expression of the gene of interest may be achieved at levels at or above endogenous levels of the gene. For example, a full-length gene may be expressed in a cell at levels that are at or above endogenous levels for that gene for that cell type. In one aspect, as demonstrated in the examples and figures herein, full-length ATM and BRCA2 may be expressed in human cells at or above endogenous levels of a cell expressing wild type ATM and BRCA2.

Expression Vectors and Cell Systems

Suitable expression vectors will be understood by one of ordinary skill in the art, and the disclosed expression vectors are intended to be exemplary and non-limiting. In one aspect, the expression vector may be a viral vector. Exemplary viral vectors include retroviral, lentiviral, adenoviral, baculoviral and avian viral vectors. Retroviruses from which a retroviral plasmid vector can be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus. A retroviral plasmid vector can be employed. The vector can be, for example, a plasmid, episome, cosmid, viral vector (as described above), or phage. Suitable vectors and methods of vector preparation are well known in the art (see, e.g., Sambrook et al., Molecular Cloning, a Laboratory Manual, 3rd edition, Cold Spring Harbor Press, Cold Spring Harbor, N.Y. (2001), and Ausubel et al., Current Protocols in Molecular Biology, Greene Publishing Associates and John Wiley & Sons, New York, N.Y. (1994)).

The vector may be used to express the gene, with or without a variant introduced, into a cell. Methods for selecting suitable mammalian host cells and methods for transformation, culture, amplification, screening, and purification of cells are known in the art. A number of suitable mammalian host cells are known in the art, and many are available from the American Type Culture Collection (ATCC, Manassas, Va.). Examples of suitable mammalian cells include, but are not limited to, Chinese hamster ovary cells (CHO) (ATCC No. CCL61), CHO DHFR-cells (Urlaub et al., Proc. Natl. Acad. Sci. USA, 97: 4216-4220 (1980)), human embryonic kidney (HEK) 293 or 293T cells (ATCC No. CRL1573), and 3T3 cells (ATCC No. CCL92). Other suitable mammalian cell lines are the monkey COS-1 (ATCC No. CRL1650) and COS-7 cell lines (ATCC No. CRL1651), as well as the CV-1 cell line (ATCC No. CCL70). Further exemplary mammalian host cells include primate cell lines and rodent cell lines, including transformed cell lines. Normal diploid cells, cell strains derived from in vitro culture of primary tissue, as well as primary explants, are also suitable. Other suitable mammalian cell lines include, but are not limited to, mouse neuroblastoma N2A cells, mouse L-929 cells, and BHK or HaK hamster cell lines, all of which are available from the ATCC. In one aspect, the gene may be expressed, as already demonstrated, in a cell line selected from HeLa (cervical cancer cell line), U2OS (osteosarcoma cell line), PE01 ovarian cancer cell line with a genetic deficiency for BRCA2, COBJT and another Fanconi anemia cell line with a genetic deficiency for BRCA2 which are SV40 Large T transformed skin fibroblasts or COBJ skin fibroblasts primary cells, COBJ EBV immortalized lymphoblasts, MCF7 breast cancer cells, and MCF10a non-transformed breast epithelial cells. The system may be utilized for a wide range of human cells—primary, hTERT immortalized, SV40 1g T transformed or cancer derived, and from various tissues of origin not necessarily limited to those listed above. Examples of packaging cells which can be transfected include, but are not limited to, the PE501, PA317, R-2, R-AM, PA12, T19-14x, VT-19-17-H2, RCRE, RCRIP, GP+E-86, GP+envAm12, and DAN cell lines. In one embodiment, the mammalian cell is a human cell. For example, the mammalian cell can be a human lymphoid or lymphoid derived cell line, such as a cell line of pre-B lymphocyte origin. Examples of human lymphoid cells lines include, without limitation, RAMOS (CRL-1596), Daudi (CCL-213), EB-3 (CCL-85), 18-81 (Jack et al., Proc. Natl. Acad. Sci. USA, 85: 1581-1585 (1988)), Raji cells (CCL-86), PER.C6 cells (Crucell Holland B.V., Leiden, The Netherlands), and derivatives thereof.

In one aspect, the gene may be expressed in a human cell, wherein the cell is genetically-deficient in the gene, or has at least about 50% deficiency in expression of the gene. In one aspect, the deficiency may be due to introduction of RNAi before or after expression of said gene. Exemplary cell types include PE01 ovarian cancer cell line with a genetic deficiency for BRCA2, COBJT, and Fanconi anemia cell lines with a genetic deficiency for BRCA2.

In one aspect, the gene may comprise a detectable epitope tag. The detectable epitope tag may be used to identify a gene product produced from said gene. Epitope tagging is a technique in which a known epitope is fused to a recombinant protein by means of genetic engineering and is known in the art. For example, by selecting an epitope for which an antibody is available, the epitope tagging allows for detection of proteins for which no antibody is available.

In one aspect, the method may comprise measuring activity of a product of the gene that is being expressed. In this way, particularly where a variant is introduced, the effect of the variant can be assayed following expression of the full-length protein. In a further aspect, the method may comprise measuring activity of a product of a gene being expressed in response to an external stimulus. The external stimulus can be any stimulus of interest, but may include one or more of DNA damage, replication stress, or oxidative stress. In one aspect, the stimulus used to illicit the damage or stress to be measured or assayed may include exposure to PARPi, MMC, or cisplatin, which induce replication stress, or ionizing radiation (IR) which induces oxidative stress.

In one aspect, a system for evaluating a gene variant is disclosed. In this aspect, the system may include a human cell type or other cell type, and a stably expressed gene as described above. The gene may codon-optimized, and may include one or more variants. The gene may further comprise one or more restriction sites that are non-endogenous, or unique, to the gene.

In one aspect, a method for determining the significance of one or more gene variants, in particular a variant suspected of contributing to disease, in particular, a cancer, is disclosed, wherein a variant is present in a codon-optimized gene, wherein the codon-optimized gene comprises at least one restriction sites that are non-endogenous, is disclosed. In this aspect, the codon-optimized gene comprises at least one restriction sites that are non-endogenous gene may be expressed using an expression vector, such as a lentivirus, to study protein structure-function or post-translational modifications.

In other aspects, the disclosed methods may guide therapy for various disease states, for example, various cancers associated with mutations in BRCA1, BRCA2, as well as the ATM may be treatable with agents such as radiation, cisplatin or PARP inhibitors that exploit deficiency of the tumor for normal DNA repair.

In one aspect, a composition comprising a lentiviral vector and a codon-optimized gene is disclosed. The codon-optimized gene may be selected from ataxia telangiectasia mutated serine/threonine protein kinase (ATM); ataxia telangiectasia and Rad3-related protein kinase (ATR); breast cancer 1, early onset (BRCA1); breast cancer 2, early onset (BRCA2); checkpoint kinase 1 (CHEK1); Fanconi anemia complementation group M (FANCM); and protein kinase, DNA-activated, catalytic subunit (PRKDC). The codon-optimized gene may comprise at least one non-endogenous restriction site. The at least one non-endogenous restriction site may be present, as described above, at an interval of not more than 1500 base pair, or at an interval of between about 100 base pairs to about 1500 base pairs, or an interval of between 250 base pairs to 1000 base pairs, or about 500 base pairs to about 750 base pairs. In one aspect, the codon-optimized gene may have a length of greater than about 6 kb.

In a further aspect, disclosed are compositions comprising a full-length BRCA2co according to Table 2 or a full length ATMco according to Table 4. In a yet further aspect, disclosed are compositions comprising a lentiviral plasmid containing a BRCA2co or ATMco variant/mutation that may be used for expression in a cell according to Table 2 or Table 4, respectively. The lentiviral plasmid may contain a benign or pathogenic variant, a variant of uncertain significance, or a deletion mutation, which may include those listed in Tables 2 or 4.

TABLE 2
Lentiviral plasmids containing BRCA2co variants/mutants
for expression in cells
Benign and Pathogenic Variants

| | | | | | |
|---|---|---|---|---|---|
| K2411T | K2472T | A2717S | K2729N | R2842H | E2856A |
| R2888C | K2950N | V3079I | Y3098H | | |
| W2626c | I2627F | L2647P | L2653P | D2723H | G2748D |
| R3052W | D3095E | | | | |

Variants of Uncertain Significance

| | | | | | |
|---|---|---|---|---|---|
| R2418G | K2434T | S2483N | L2587F | P2589S | V2610M |
| W2619S | H2623R | I2628T | A2643V | R2651T | T2662K |
| I2664M | T2681R | S2709G | V2739I | I2752F | A2770D |
| M2775T | R2784W | R2784Q | S2810G | G2812E | V2818I |
| R2842C | S2922N | Q2925H | A2942T | L2972W | Y3035C |
| K3059N | D3064Y | C3069F | Y3092C | | |

Deletion Mutants for Domain Mapping

| | | | | | |
|---|---|---|---|---|---|
| Δ6K-53K | Δ54N-150H | ΔV151- | Δ248A- | Δ379G- | Δ503K- |
| | | 1247 | 378S | 502K | 615N |
| Δ616C- | ΔV746- | Δ878D- | ΔN1002- | ΔS1560- | ΔN2101- |
| 745K | P877 | 1004S | 11556 | L2092 | P2276 |
| ΔV2280- | ΔL2396- | ΔI2672- | ΔV2815- | ΔL3055- | ΔI3107- |
| N2390 | S2667 | S2810 | Y3049 | A3102 | S3250 |
| 3267KR- | ΔL3271- | ΔS3319- | 3389SL- | | |
| NG NLS1 | K3313 | S3368 | XX | | |

In a yet further aspect, also disclosed is a cell according to Table 3 or Table 5, comprising any of the sequences of Table 2 or Table 4.

TABLE 3
Cells Expressing BRCA2co

| | | | | |
|---|---|---|---|---|
| HelaS3 | FA-D1 LCLs | FA-D1 | FA-D1 Lg T | T47D |
| | | primary fibs | fibs (2) | |
| MCF7 | MCF10a | U2OS-DR | mDA-mB-231 | |

TABLE 4
Lentiviral plasmids containing ATMco variants/mutants
for expression in cells
Benign and Pathogenic Variants

| | | | |
|---|---|---|---|
| S1983N | I2030V | G2287A | L2332P |
| R2032K | R2227C | S2394L | V2424G |
| S2716F | | | |

Variants of Uncertain Significance

| | | | |
|---|---|---|---|
| R2034Q | R2392W | R2719H | T2743M |
| I2776T | G3029D | L3048V | |

Deletion Mutants for Domain Mapping

| | | |
|---|---|---|
| Δ90-143 | Δ1287-1516 | Δ2353-2553 |

Post-Translational Modifications and Kinase-Dead Mutations

| | | | |
|---|---|---|---|
| S1981A | C2991L | K3016R | D2870A/N2875K |

TABLE 5
Cells Expressing ATMco

| | | | |
|---|---|---|---|
| A-T LCLs | A-T Lg T | HelaS3 | U2OS-DR |
| | fibs (2) | | |

It should be noted that for any of the above described methods or compositions, the codon-optimized gene or variant may be expressed at a level at or above endogenous levels of a wild-type version of the codon-optimized gene. In other words, for the disclosed expression systems, expression of the codon-optimized gene may easily be obtained, at levels that may be in excess of that normally observed in a cell type that normally expresses the wild-type gene.

### Examples

The following non-limiting examples are provided to further illustrate embodiments of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the invention.

ATM

The function(s) of most regions of ATM, including known domains such as the FAT domain, and the impact of VUS, remains largely undefined. Further, to the best of Applicant's knowledge, no validated system to functionally classify the effects of ATM variants on disease risk currently exists. This is particularly important because, as noted by the ENIGMA consortium, genetic data are generally not sufficient for the classification of missense VUS, since they are typically rare. Disclosed is an innovative system to functionally characterize ATM VUS and mutations which has two key features 1) the innovative utilization of a lentiviral vector to express ATM variants based upon a codon-optimized cDNA and streamlined generation of expression constructs; and 2) functional assays based on correction of altered cellular responses to DSBs in ATM-deficient cells, as demonstrated in referenced figures.

Next-generation sequencing (NGS) technologies are identifying large numbers of variants in an assortment of genes. Understanding the effect of these variants on features such as protein function, disease risk, prognosis and response to therapy remains very challenging, however. Indeed, for numerous genes, many of the variants that are identified in genetic screens are variants of uncertain significance (VUS). This is especially the case for missense VUS, in part because they frequently are rare, meaning that classic genetic segregation analyses are underpowered. Additionally, greater numbers of VUS are generally detected in large genes (such as ATM or BRCA), since the number of VUS identified intends to increase linearly with the length of the DNA.

ATM protein expression has been problematic, in part due to large gene size and poor mRNA quality. This has greatly restricted studies to characterize the effects of ATM variants and the roles of different regions of ATM. Additionally, no rigorously validated system, nor one calibrated for sensitivity and specificity, has previously been established to classify ATM VUS. The disclosed methods for the rapid and efficient expression of full-length human ATM in ATM-deficient cells using a lentiviral vector and a codon-optimized cDNA can be used for efficient expression and characterization of ATM., which is versatile because this system can be utilized for expression in human cell types depending on the particular need. Importantly, by synthesizing variants in fragments of ATM which are then inserted into the expression vector, the modular approach disclosed herein is rapid and capable of evaluating ATM VUS on a large scale.

ATM VUS may be characterized using DSB-related assays by testing benign and pathogenic standards that have previously been defined based on clinical and genetic criteria. Any portion of the gene may be characterized, but the methods may be particularly useful in characterizing certain regions such as the missense ATM VUS of the C-terminal FATKIN region, for example, which contains the kinase domain and key regulatory elements, and which is where the most known pathogenic missense ATM variants reside. Functional assay results may be combined with a multifactorial analysis, along with clinical and genetic data, for robust predictions of cancer risk associated with missense ATM variants. Another limitation to understanding the effects of variants, and the role of ATM in preventing disease, is a need to better define the roles of distinct regions of ATM, which is largely unknown. By expressing mutants that delete regions throughout the protein, the disclosed methods may be used to test the effects of pathogenic variants on binding to the NBS1 activator, for example, and may be used to interpret the 3-dimensional structural effects of pathogenic variants in the FATKIN region. As a result, Applicant now provides novel methods useful for characterization of VUS that may be used to dramatically improve understanding of ATM function.

Given the diverse biological functions of ATM, the association of deleterious ATM gene mutations with human disease, and the large number of known ATM VUS, there is a need for addressing the issue of variant classification. The ATM gene encodes the ATM protein kinase, which by phosphorylating various substrates, is a key regulator of the cellular response to DNA double-strand breaks (SDBs) and coordinates apoptosis, DNA repair, and cell cycle telangiectasia (A-T) patients, resulting from biallelic germline mutations in ATM. Clinical features of A-T patients include neurodegeneration, immune dysfunction, radiosensitivity, and a predisposition to lymphomas, leukemias, and other cancers. Additionally, ATM has been identified as a breast (moderate penetrance) and pancreatic cancer susceptibility gene based on increased risks associated with heterozytosity for germline ATM mutations. ClinVar, a database that lists variants, has over 2,480 distinct germline missense ATM VUS found in A-T and/or cancer patients. This limits the utility of genetic screens in guiding clinical care.

In the many cases where classic genetic approaches are not sufficient to classify ATM variants, functional assays provide a promising alternative. Importantly, no validated approach for the systematic functional characterization of ATM VUS has been established in the scientific literature. Difficulty with efficient expression of full-length ATM has been a strong impediment to such studies. To overcome this obstacle, Applicant utilizes synthetic biology to rapidly generate VUS identified in patients, and a codon-optimized ATM cDNA (ATMco) to express the VUS in ATM-deficient human cells.

ATM is a serine threonine protein kinase that, by mediating DNA damage signaling, has a central role in the cellular response to DNA double-strand breaks (DSBs). DSBs induced by ionizing radiation (IR) and other agents are among the most genotoxic DNA lesions. The broad importance of ATM is demonstrated by the association of ATM mutations with human disease. Ataxia-telengiectasia (A-T) is a multi-system disease caused by biallelic mutation of ATM, and is typified by neurodegeneration, immunodeficiency, radiosensitivity and a predisposition to lymphocytic malignancies. A-T patients may also display features such as growth retardation, premature aging, insulin resistance, and developmental abnormalities of reproductive organs. Heterozygous germine loss-of-function mutations in ATM also increase the risk of developing breast and pancreatic cancer, and perhaps other malignancies such as prostate and stomach cancer. While their role in driving disease is largely unknown, somatic mutations in ATM have been observed in many cancer types.

By phosphorylating numerous proteins, including NBS1 and CHK2, ATM coordinates apoptosis with cell cycle regulation and DNA repair, thereby maintaining genome stability. Central to its diverse roles in mediating the DSB responses, ATM activates a partner kinase, CHK2, by phosphorylating it at Thr68. Another key ATM-dependent signaling event is feedback phosphorylation of NBS1, which has a role as a damage sensor that activates ATM. ATM also regulates the G2 checkpoint, which delays progression into mitosis in response to agents that induce DSBs. While ATM has additional roles in other processes, such as transcription, redox homeostasis and regulation of mitochondria DSB-related roles of ATM are the clear choice for functional assays used here to evaluate the effects of ATM variants. The reasons include 1) the central function of ATM in the DSB response; 2) cells from A-T patients display cellular sensitivity and chromosomal instability in response to DSBs; 3) unlike ATM's other roles, defects in the DSB response may contribute to each of the clinical manifestations of A-T; 4) pathogenic variants/mutations of ATM are associated with defects in DSB responses.

Human ATM is a very large protein (about 350 kDA, 3056 amino acids) that is very difficult to express. Previous studies have established that full-length ATM can complement radiosensitivity, and IR-induced G2 checkpoint defects and deficient signaling in cells from A-T patients, providing a basis for cellular assays to characterize ATM variants. Only seven domains have been identified in this huge protein, mostly located at the C-terminus. However, the detailed functions of most of these domains is still poorly characterized. For example, the specific domains that regulate G2 checkpoint function are still unknown.

ATM variants of uncertain significance (VUS) are rapidly being identified in genetic screens. In fact, in two recent NGS studies, ATM was the gene found to harbor the most VUS on multi-cancer gene panels that included BRCA1 and BRCA2. ATM variants that truncate the protein are, in general, considered clearly pathogenic since critical functional domains are located in the C-terminal FATKIN region. In contrast, the impact of missense ATM variant on protein function and disease is unclear. For example, about 2590 distinct germline ATM variants identified in genetic screens are currently listed in ClinVar, a public database of human variants. Over 84% of these ATM VUS are missense variants (about 2480) observed in A-T patients and/or individuals at risk for a hereditary cancer syndrome. In ClinVar and a recent study (Decker B, Allen J, Luccarini C, et al. Rare, protein-truncating variants in ATM, CHEK2 and PALB2, but not XRCC2, are associated with increased breast cancer risks. J Med Genet. 2017; 54(11):732-741), missense VUS distribute evenly throughout ATM. These figures underestimate the number of missense ATM VUS, since there are other databases and not all clinical testing laboratories contribute to ClinVar. Genetic tests alone, such as co-segregation of variants with cancer in affected family members, are generally insufficient to classify ATM missense VUs.

Strikingly, more than 35% of the individual missense ATM VUS listed in ClinVar, occurring throughout the protein have been reported both in A-T patients and individuals tested for cancer susceptibility. While missense variants are observed less frequently in A-T patients than truncating mutation in ATM, the presence of biallelic alterations in ATM in A-T patients alone may not be sufficient for classifying missense variants as pathogenic, For example, missense variants in A-T patients may be present in cis on a particular allele.

Genetic screens for ATM mutations are becoming more prevalent. While less well established than for BRCA1/2, the detection of deleterious germline mutations in ATM can potentially be utilized as a basis for increased surveillance to enable early cancer detection, and for implementing surgical measures that can reduce risk and cancer-related mortalities. However, because prophylactic interventions can have a huge impact on the quality of life and health, and can be expensive, their election should be based upon accurate determinations of variant pathogenicity. In addition to modulating cancer risk, germline (and somatic) ATM mutations can potentially modulate the response of various cancer types to treatment with platinum compounds and poly ADP ribose polymerase inhibitors. Detection of the mutational status of ATM may also permit targeting of ATM-proficient and deficient tumors with ATM and ATR inhibitors, respectively. Additionally, biallelic missense ATM mutations can cause a mild form of A-T with slower progression. Thus, classification of missense ATM may have prognostic value in mild/unrecognized forms of A-T

The high frequency of unclassified ATM variants, and uncertainty about the function of most regions of ATM, severely limits the utility of genetic screening to predict disease risk. It is therefore paramount to develop assays that can reliably classify large number of ATM VUS, including missense alterations. This will empower genetic screens and increase understanding of the impact of genetic variation.

As a basis for classifying VUS, the International Agency for Research on Cancer Working Group (IARC) developed a system that includes genetic data (such as co-segregation), tumor histopathology and sequence conservation across species. It also includes the properties of mutated residues analyzed using bioinformatics tools, which have not proven sufficiently reliable as a stand-alone method for classifying VUS. The IARC defines class 1 variants as non-pathogenic (benign; probability of pathogenicity, p<0.01)), class 2 as likely benign (probability>0.01 but <0.05), class 4 as likely pathogenic (probability<0.99 but >0.95) and class 5 as pathogenic (probability>0.99), with class 3 remaining unclassified (VUS). However, even this multifactorial system frequently cannot classify rare variants identified in genetic screens.

Functional assays provide additional information which can potentially be utilized to classify variants, as recognized by the Evidence-based Network for the Interpretation of Germline Mutant Alleles (ENIGMA) consortium. ENIGMA has outlined a strategy for validating functional tests of VUS based on known IARC Class 1/2 (benign/likely benign) and 4/5 (likely pathogenic/pathogenic) variants. Such validation enables consideration of clinical and genetic data together with the results of functional tests. However, there currently is no well-established system for the functional analysis of ATM VUS. Applicant has developed a functional assay with 1) a high degree of sensitivity (correct identification of pathogenic variants) and 2) a high degree of specificity (correct identification of benign variants).

The very large size and low mRNA quality (not shown) of ATM cDNA has been a major barrier to stable expression of ATM. To surmount this challenge, Applicant has developed a unique system that has the following unprecedented capabilities and features: 1) efficient expression based upon a codon-optimized (“co”) cDNA, including incorporation of codons corresponding to tRNAs that are more abundant in human cells, which is unprecedented; 2) a lentiviral vector (while the codon-optimized cDNA can potentially be used with other plasmids or viral vectors, lentivirus may be most effective due to its capacity to package large amounts of DNA and ability to infect non-cycling cells); 3) a rapid process for generating expression constructs that contain variants that is greatly streamlined by incorporating approaches utilized in synthetic biology (specifically, variants for a particular fragment can be batch synthesized then introduced into the codon-optimized cDNA utilizing unique restriction sites contained once in the codon-optimized cDNA but not the original, naturally-occurring cDNA). Importantly Applicant has demonstrated that ATMco cDNA can efficiently express full-length ATM in human cells. FIG. 10 depicts an exemplary codon optimized ATM (ATMco) and FIG. 2 depicts an exemplary scheme for the disclosed methods.

Synthesis and modular insertion of variant-containing fragments (−500-1,500 bp) of ATM is feasible because ATMco is engineered to contain unique restriction sites not present in the naturally occurring cDNA (many appear as a direct result of the change to optimized codons). Thus, Applicant's process for constructing expression vectors removes multiple time-consuming steps. As site-directed mutagenesis is not performed on the expression vector, the only mutation is the variant to be tested. To maximize disease-relevance, and to achieve an isogenic background to facilitate comparison of variants, ATMco cDNA may be used to stably express full-length ATM, harboring variants, in an ATM-deficient cell from an A-T patient (or in other cell types, as needed). Full-length ATM is used for assays given that domains (or regions) throughout the protein may be necessary for wild-type (WT) levels of activity. The novel system may be used to characterize ATM missense VUS and to define the function(s) of post-translational modifications or functional domains throughout ATM.

The disclosed systems allow for stable and efficient expression of full-length ATM using a lentiviral vector system and codon-optimized cDNA. This, in turn, provides a streamlined and error-free process for rapid generation of expression constructs containing variants, and customized generation of deletion mutants to test region-specific functions, based upon synthesis of fragments and insertion into ATMco.

Results

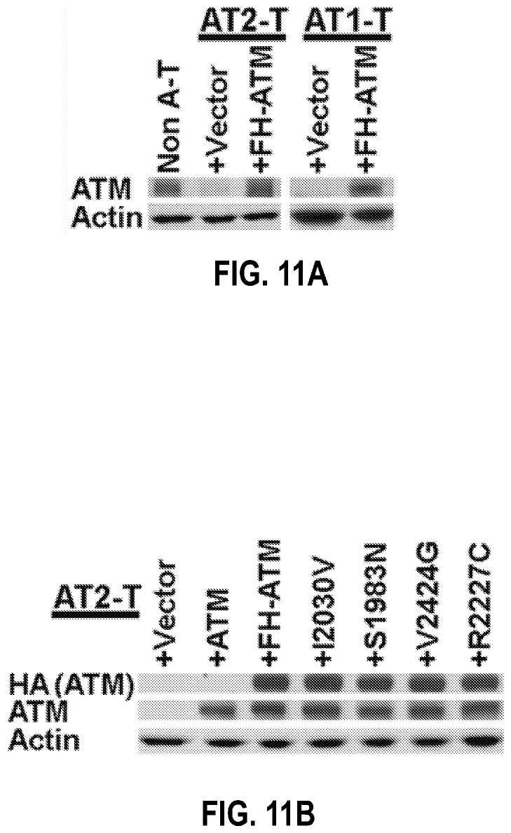

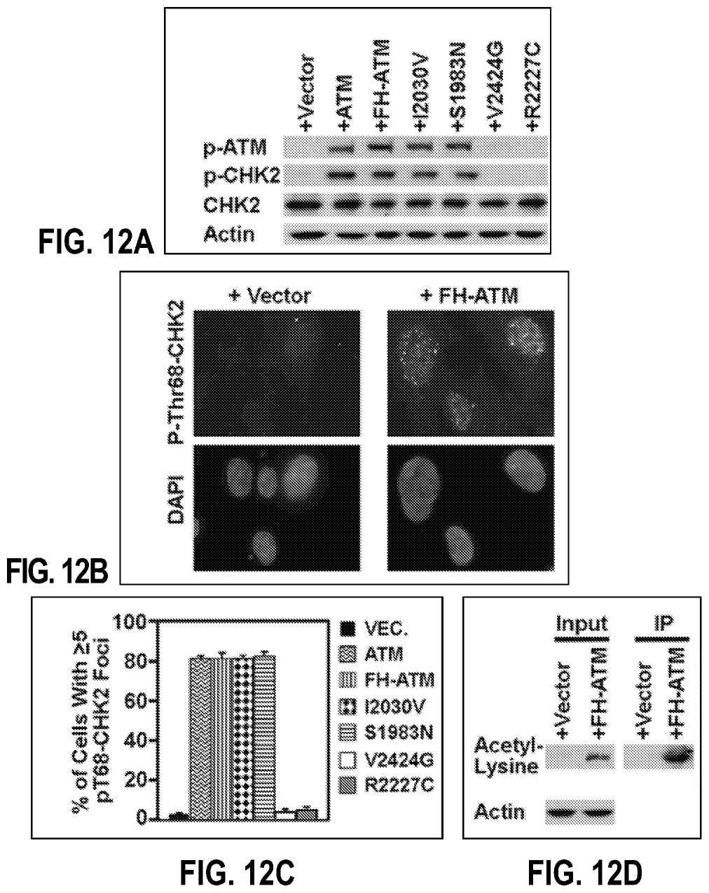

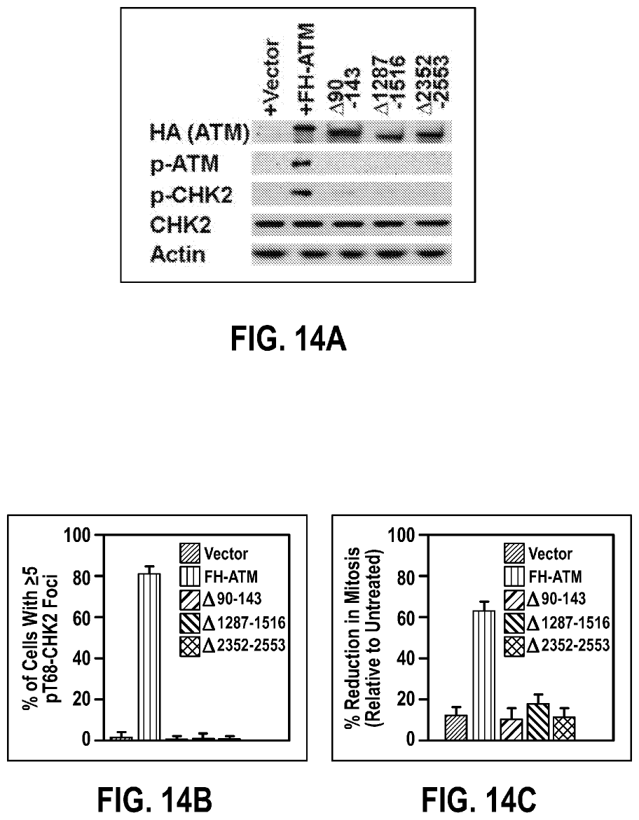

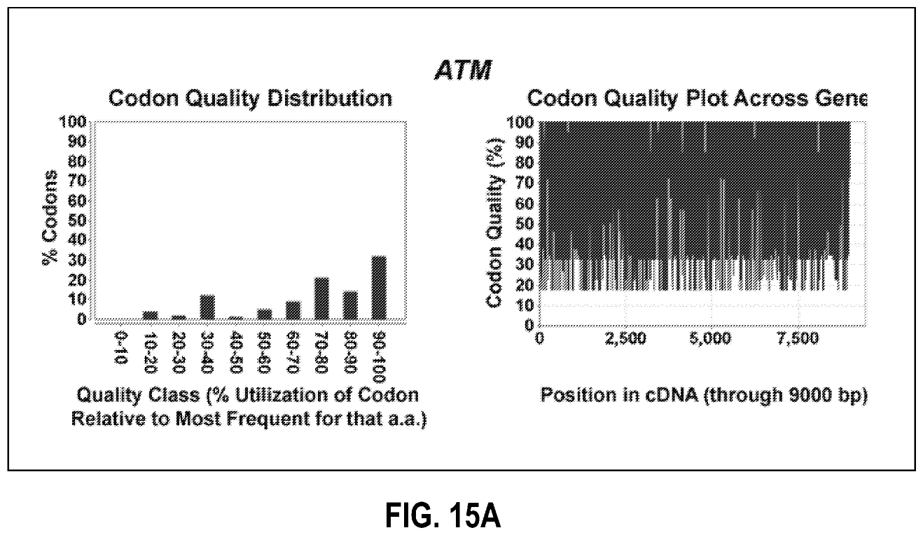

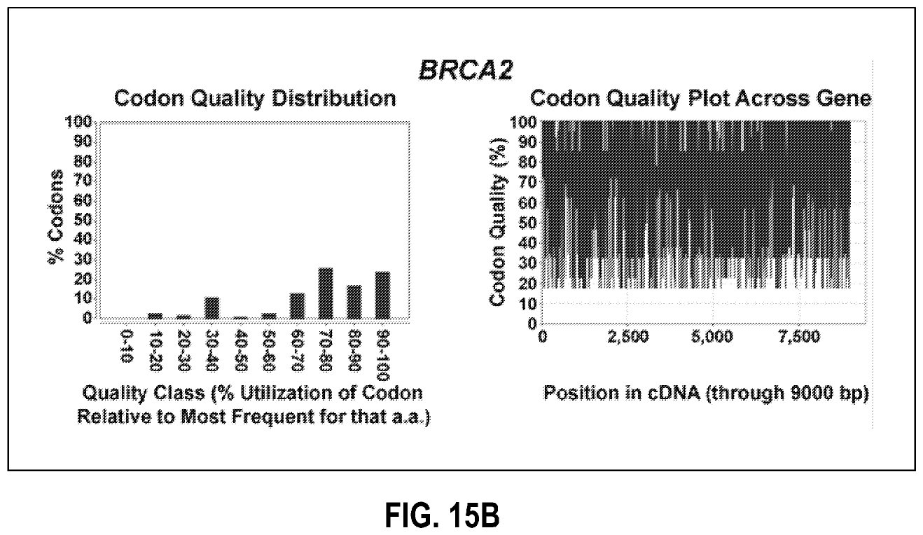

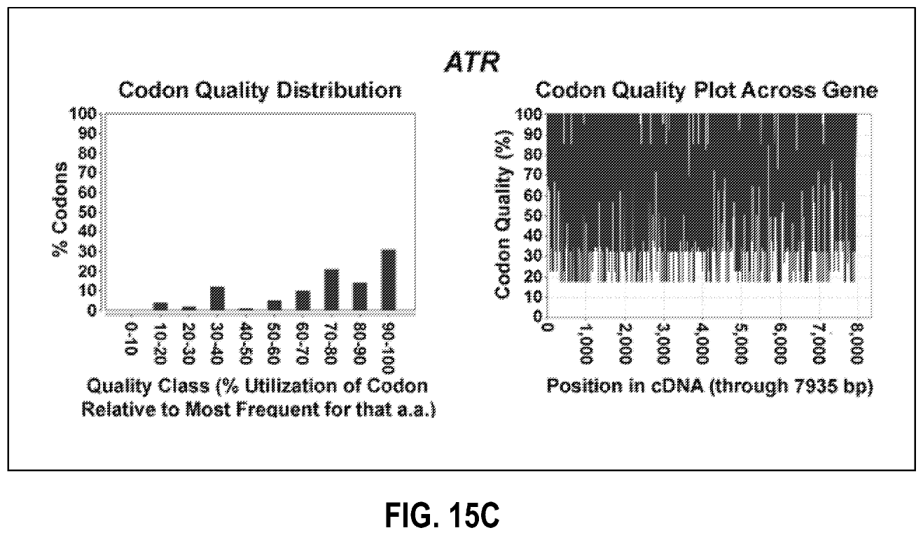

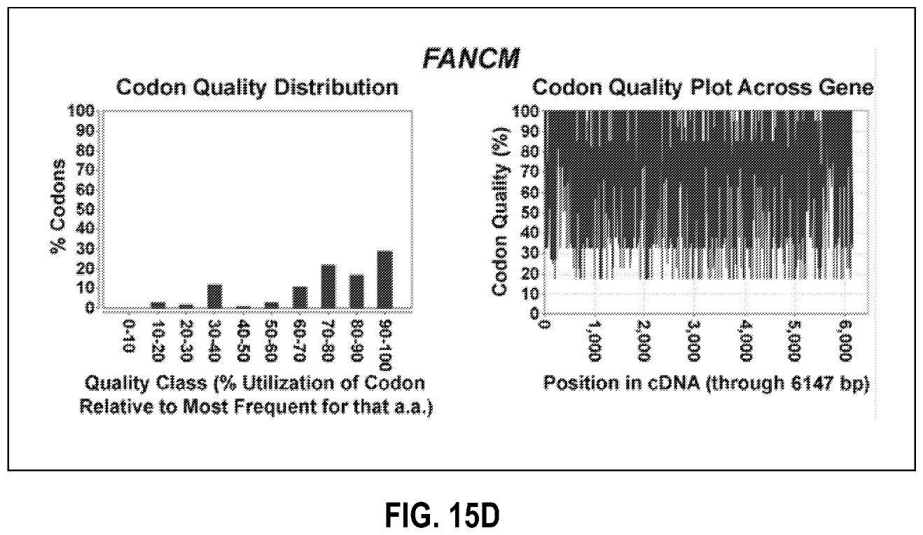

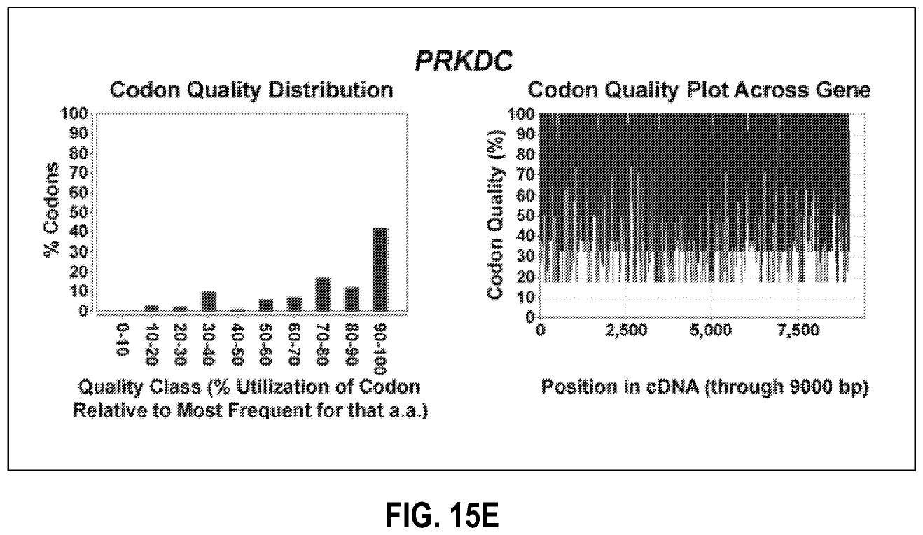

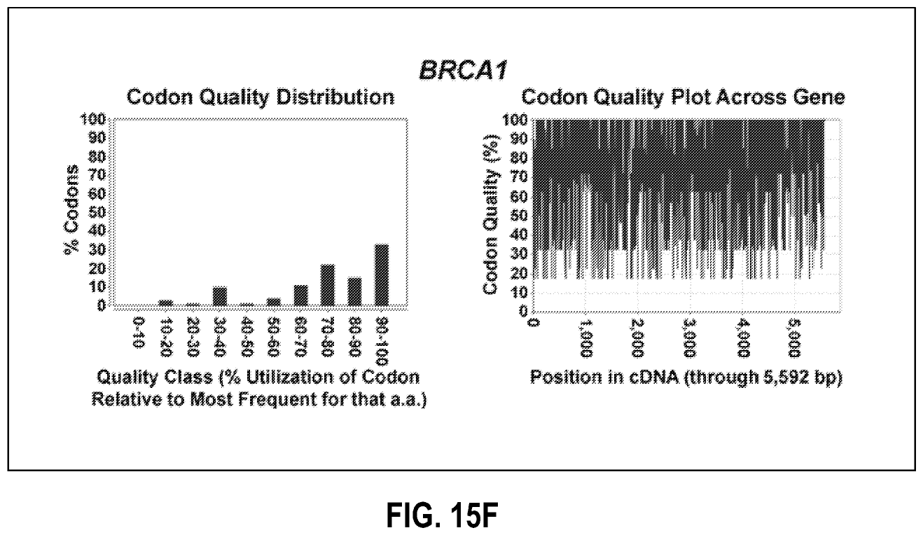

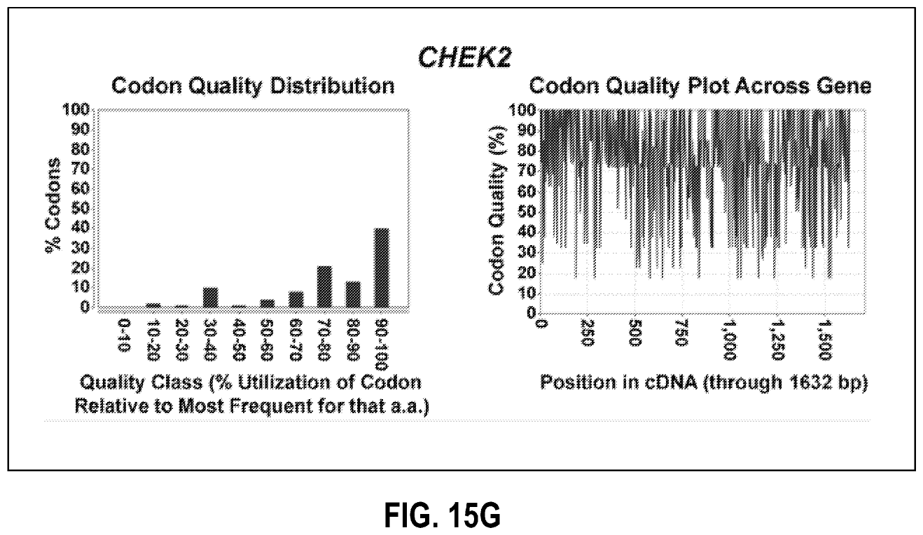

FIGS. 11-14 use cells from A-T patients [GM15786, GM02052-T (AT1-T), and AT2-1] with biallelic ATM mutations. AT1-T cells have near complete loss of ATM due to homozygosity for a truncating mutation in exon 1. ATMco contains a Flag-HA (FH) epitope tag at its N-terminus, unless noted otherwise. Benign/likely benign variants (IARC Class 1 & 2) with a probability of pathogenicity of <0.05 and likely pathogenic/pathogenic variants (Class 4 & 5) with a probability of >95% are employed. Given the central function of ATM in it, and because cells from A-T patients are defective for it, the assays in FIGS. 12-14 are related to the DSB response.

A novel cDNA-based system for efficient expression of ATM in human cells. Efficient and stable expression of human ATM cDNA in cells has been problematic, until now. Applicant has used a codon-optimized (co) ATM cDNA to stably express full-length ATM in 3 human ATM-deficient and 2 ATM-proficient cell types (data not shown). In all 3 ATM-deficient lines, functional correction of the ATM deficiency was verified using DNA repair-related assays. Levels of expression of ATMco were similar to that of endogenous ATM in normal (non A-T) control cells (FIG. 11, A). Expressed ATM is full-length as demonstrated by detection with an antibody recognizing the C-terminus of ATM (amino acids 2577-3056) and by a size similar as ATM in non A-T control cells. Importantly, the expression system confers reproducible and comparable levels of expression of WT ATMco, and of 2 benign and 2 pathogenic variants. Thus, the inventive expression system uniquely enables the systematic analysis of ATM VUS, ATM post-translational modifications and definition and testing of ATM functional domains.

Functional assays based upon expression of ATMco in cells that are genetically-deficient for ATM. ATM-deficient cells display defective DNA damage signaling, as seen in AT1-T cells transduced with empty vector. However, ATMco is autophosphorylated at S1981 and corrects deficient CHK2 phosphorylation at T68 (pCHK2) in cells exposed to ionizing radiation (IR), as measured on Western blots (FIG. 12A). Further, ATMco rescues damage-induced assembly of pCHK2 into nuclear DNA damage foci (FIG. 12, C). Two benign missense variants (p.I2030V and p.L2332P) and two pathogenic missense variants (p.R2227C and p.V2424G) were proficient and deficient, respectively, for pCHK2, both on Western blots and using foci assays. It should be noted that slight variability in ATM expression does not affect the assays. Given the position of acetylation at the extreme C-terminus of ATM, this further demonstrates that full-length ATM is being expressed.

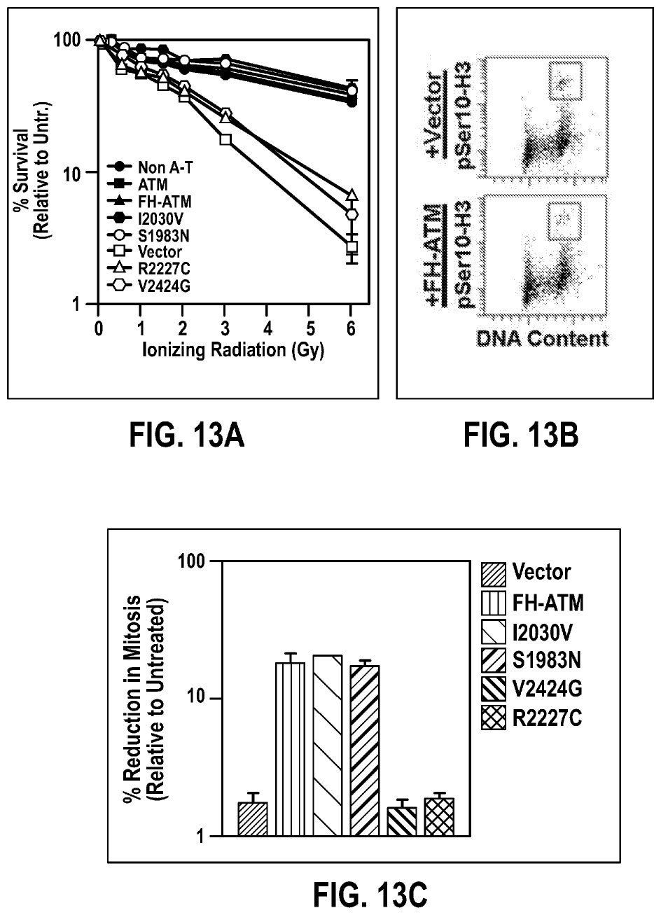

Another readout for ATM function is cellular resistance to IR. ATMco, with or without a Flag-HA epitope tag, confers cellular resistance of ATM-deficient cells to IR that is indistinguishable from non A-T cells. Codon-optimization and the epitope tag also did not alter ATM-dependent damage signaling, and therefore can be utilized to reliably test ATM VUS. Two benign missense variants restored cellular resistance of ATM-deficient cells to IR, while 2 pathogenic missense variants did not (FIG. 13, A).

Another key function of ATM is in mediating G2 checkpoint arrest in response to DNA damage. ATMco corrects the G2 checkpoint defect in ATM-deficient cells treated with IR by decreasing levels of mitosis. Further, two benign missense ATM variants similarly corrected the checkpoint defect, but two pathogenic variants did not (FIG. 13C). Because the cDNA-based system can readily distinguish variants associated with undetectable function or full loss of ATM function using a variety of DNA damage response assays, it is well suited to the functional characterization of ATM VUS.

BRCA

A substantial proportion of breast, ovarian, and pancreatic cancers are due to a genetic mutation. Using ovarian cancer as an example, there are currently 11 distinct demonstrated or suspected ovarian cancer genes that cause this disease in humans when inactivated by mutations. These mutations can either be inherited from a parent or can occur spontaneously. Importantly, mutations in these ovarian cancer genes can potentially occur in any woman, so current guidelines for care recommend genetic screens to identify women with such mutations. This is important because identification of inactivating mutations can be utilized by genetic counselors and healthcare providers to enable preventative measures and/or to select treatment options tailored to that patient's tumor.

Screens for mutations in cancer genes are based upon sequencing the genetic material (DNA) from patients. Additionally, screens of family members can identify individuals with inherited (germline) mutations that increase their risk of developing cancer, including breast and ovarian cancer. Importantly, information from such screens can save lives by enabling earlier detection and/or prevention of cancer. Screens for mutations are also very important for treating cancer, once diagnosed. Drugs that are utilized to treat cancer typically damage the genetic material, including genes. Most of the known or suspected breast and ovarian cancer genes have a role in limiting this damage by encoding for proteins that repair it. Platinum compounds, which are a mainstay in the treatment of ovarian cancer and many other cancers, are an example of how mutation can modulate the response to treatment. Platinum compounds are more effective in women with either inherited or spontaneous (somatic) mutations in many of the breast and ovarian cancer genes. As another example of how deleterious mutations can be exploited, poly (ADP-ribose) polymerase (PARP) inhibitors are targeted therapeutics that can selectively kill tumor cells with a genetic defect in these same genes. Thus, identifying patients with either germline or somatic mutations in cancer genes, which can potentially occur in any woman, helps guide treatment of breast, ovarian and potentially other cancers.

BRCA2 along with BRCA1 are the two genes most often mutated in patients with hereditary ovarian cancer, and about 28% and 47% of hereditary ovarian cancer is due to mutations in BRCA2 and BRCA1, respectively. BRCA2 and BRCA1 are also the major breast cancer genes, consistent with many pedigrees that carry heterozygous germline mutations in these genes having histories of both ovarian and breast cancer. Overall, carriers of BRCA2 mutations have a >20% lifetime risk of developing ovarian cancer. Importantly, the age of onset is lower than in the general population and resulting tumors tend to have a higher grade in carriers of BRCA2 mutations. Somatic mutations in genes such as BRCA2 and BRCA2 are also frequently seen in sporadic cases of ovarian cancer.

Unfortunately, there is a basic problem with screening for genetic changes in BRCA2 and other genes. This problem is that only some mutations cause cancer, while others are harmless. Thus, there is a need to distinguish which mutations have the capacity to cause cancer from those which do not. In fact, such predictions currently cannot be made for most mutations of BRCA2. To address this critical issue, Applicant has developed a novel system for testing BRCA2 mutations in order to improve prevention and treatment of cancers associated with germline or somatic mutations in BRCA2 and related genes. The disclosed methods can be used to generate and express mutants of BRCA2 in cells that are already genetically defective for BRCA2. Expression of a normal copy of the BRCA2 protein corrects defects in DNA repair in these cells, and allows for the determination of which mutants of BRCA2 have a compromised ability to repair damage to the genetic material. The disclosed methods can be used to assess previously unclassified mutations in BRCA2 found in breast, ovarian, pancreatic and potentially other cancer patients, for which the risk is currently unknown. These assays can be used to predict an increased risk for developing breast and ovarian cancer in women who harbor harmful mutations. This information can then be utilized to guide cancer prevention measures, including surgical measures, as well as counseling concerning environmental and lifestyle hazards that increase cancer risk in these patients. The systems may also be useful for determining whether particular mutations in BRCA2 may lead to more effective killing of cancer cells by PARP inhibitors or other compounds. This information can then be utilized to predict which patients are most likely to benefit from treatment with PARP inhibitors (or other compounds or classes of compounds, as determined using the disclosed methods), based on mutations they harbor, and patients more likely to benefit from other treatments. It should be noted that most PARP inhibitors are FDA approved only for patients with deleterious mutations in HR genes.

Prior to Applicant's invention, there has been no system for determining the effect of mutations in several other cancer genes that are related to BRCA2 based upon function in DNA damage responses related to DNA double-strand breaks, DNA interstrand crosslinks and replication stress, including ATM, ATR, BRCA1, CHEK2, and FANCM. BRCA1, in particular, is the most frequently mutated gene that drives breast and ovarian cancer, and BRCA1 protein is difficult to stably express in an efficient manner. The disclosed methods further provide assays for unclassified mutations of these breast/ovarian cancer genes for assessing defective DNA repair and increased cellular sensitivity to potential therapeutic agents. For example, the methods may be used to determine mutations and the effect on sensitivity to a PARP inhibitor such as olaparib. The disclosed methods may also be used to predict how mutations in these genes affect cancer risk.

In the case of BRCA2, current systems have a limited capacity to assess the risk of mutations that are detected, but Applicant's system has a much greater short-term and long-term capacity to assess mutations. Results from in vitro tests can be utilized by genetic counselors and clinicians to guide the care of patients who are at an elevated risk of developing, or who already have, breast or ovarian cancer and potentially other cancers.

The BRCA2 protein, and related proteins, have an important role in DNA repair and in the maintenance of genome stability. BRCA2 is a tumor suppressor gene that has well-known roles in DNA repair by homologous recombination (HR). It controls the oligomerization of the RAD51 recombinase into a nucleoprotein filament with single-strand DNA, thereby initiating HR.

Human BRCA2 is a very large protein (˜385 kDa) and has multiple characterized domains, which are involved in mediating HR. These include eight BRC repeats (interspersed between amino acids 1008-2082) which bind to RAD51. Additionally, a helical domain (amino acids 2482-2668) and 3 C-terminal OB-folds bind DNA (amino acids 2670-3102). Recently, a N-terminal DNA binding domain (DBD) has also been identified and there is also an additional RAD51-binding domain at the C-terminus of BRCA2 (amino acids 3260-3314). Variants of BRCA2 occur throughout the protein, both within these identified domains and in other regions. In addition to the need to characterize the effects of variants throughout the protein on cancer risk and response to therapy, the function of large parts of the proteins and of many post-translational modifications is currently unknown.

Mutations in BRCA2, and other DNA damage response-related genes, can drive the development of cancerous cells through increased levels of genome instability. Determination of the risk of developing cancer, based upon specific germline mutations harbored by each patient, is critically important for genetic counseling, for early detection, and for cancer prevention that includes surgical measures. Additionally, many chemotherapeutic agents, including cisplatin, kill tumor cells due to the induction of DSBs and other forms of DNA damage that are repaired by homologous recombination (HR). As a result, mutation of BRCA2 has been found to be linked to increased responsiveness of ovarian tumors, and other types of cancer, to platinum compounds and/or to better overall survival. Further, inhibitors of poly ADP ribose polymerase (PARP), which exploit defects in BRCA2 and other HR proteins to induce synthetic lethality, have proven most effective in patients that harbor deleterious mutations in the corresponding genes. Thus, prediction of whether a particular mutation that the patient may harbor, either germline or somatic, is deleterious, is also a key to personalized treatment, termed precision medicine, that can be tailored to exploit defective HR in the tumor using PARP inhibitors.

The clinical significance of clear loss of function mutations, such as those that result in truncated proteins, is often readily interpreted. The clinical importance of missense changes/unique variants is often quite difficult to determine, however. In particular, clinical and family histories are frequently insufficient for genetic classification of specific missense variants based on co-segregation of the variant with cancer in afflicted family members. While functional tests that are based upon expression of the mutant protein provide a potential alternative for stratifying variants of uncertain significance (VUS), to date such approaches have had a limited impact for BRCA2. This stems, at least in part, from the extremely large size of the encoded protein (˜385 kDa) and difficulty expressing WT and variant forms, since this is the basis for functional assays.

The disclosed methods may be used for screening for mutations which predispose women to breast and ovarian cancer, and various cancers in men and/or women including cancers of the prostate and pancreas, and utilization of the results for cancer prevention. Determination of the risk for developing cancer, based upon identifying specific germline mutations present in each patient using DNA sequencing, can be lifesaving. Such screening can provide guidance for increased surveillance that enables early detection and prophylactic measures. In particular, surgical removal of the breast or ovaries (oophorectomy), often along with removal of the fallopian tubes, is an important means for reducing the risk of developing cancer in carriers of BRCA1 or BRCA2 mutations. Oophorectomy, in particular, is a radical procedure that sends women into menopause, but results in an 80-95% reduction of the risk of developing ovarian cancer in pre-menopausal women and also reduces breast cancer risk. As this surgical procedure has huge implications for the quality of life and health of these women, however, such decisions should be based upon highly reliable predictions of whether or not a particular variant detected in genetic screens is deleterious. Lifestyle changes and decreased exposure to certain environmental factors can reduce cancer risk, but determining the pathogenicity of BRCA1/2 variants remains necessary to delineate hereditary risk in carriers.

The disclosed methods may be used to screen for mutations which predispose women to breast and ovarian cancer, and potentially other cancers, and to guide treatments for cancer. As a basis for personalized, or precision, medicine, genetic screens for germline or somatic mutations in BRCA2, and other ovarian cancer genes related to cellular responses to DNA damage, can also potentially be used to guide cancer therapy. For example, deleterious mutations in BRCA2 can increase clinical response to chemotherapeutic agents such as cisplatin. Further, inhibitors of poly ADP ribose polymerase (PARP) exploit the loss of BRCA2 function in the tumor cells of patients and selectively kill them. While niraparib has demonstrated activity against a subset of non-BRCA ovarian cancers in the absence of compromised HR, and despite the fact that reversion mutations in HR genes can drive acquired resistance of tumors to PARP inhibitors (PARPi), PARPi have proven most effective in patients with deleterious BRCA1/2 mutations. As such, in most instances, PARPi have been approved by the US Food and Drug Administration (FDA) for treating advanced ovarian cancer in patients with germline mutations in BRCA1/2 but not patients that harbor BRCA1/2 VUS. It should be noted that treatment with PARPi can cause severe adverse effects. For these reasons, predictions of the potential effects of particular VUS of BRCA2, and related cancer genes, on the response to PARPi is greatly needed to guide selection of therapeutic options. The disclosed methods can be used to both predict the effect of somatic VUS on the response to PARPi, but also the effect of germline VUS, which are being rapidly identified in genetic screens. Predicting their effect on response to PARPi will better empower these screens.

While heterozygous mutation of BRCA2 and BRCA1 is associated with an increased risk of developing breast, ovarian and other cancers, biallelic mutation of these genes causes the D1 and S subtypes, respectively, of the rare childhood disorder Fanconi anemia (FA). FA is characterized by chromosome instability, congenital anomalies, and a predisposition to acute myeloid leukemia and solid tumors. Thus, further understanding mutations and the functional effect is also useful to potential risk assessment and treatment of FA.

Classification of Variants of Uncertain Significance (VUS).

While truncating mutations may delete functional domains and thereby be clearly pathogenic, the classification of missense variants is more difficult. Genetic tests, such as co-segregation of the variant with cancer in affected family members, are a powerful basis for classifying variants. But as exemplified by BRCA2 missense variants, most of which are rare, genetic tests alone are generally insufficient to classify these variants. As a response, the multifactorial 5-tier classification system has been developed by the IARC as described above. This system can be utilized to classify VUS based upon co-segregation with cancer in families, co-occurrence with previously identified pathogenic mutations and tumor histopathology, combined with an analysis of sequence conservation across species and the properties of mutated residues. IARC Class 1 and Class 2 designate non-pathogenic (probability less than 0.01) and likely non-pathogenic variants (probability>0.01 but <0.05), respectively. In contrast, Classes 5 and 4 represent pathogenic (probability>0.99) and likely pathogenic variants (probability<0.99 but >0.95), respectively. Variants with intermediate probabilities (>0.05 but <0.95) remain unclassified and are designated as Class 3, largely due to the insufficient availability of family information. Also, neither bioinformatics tools to analyze residue changes encoded by a particular mutation, nor mutational signatures identified in carriers of BRCA1/2 mutations, has proven reliable or sufficient as a stand-alone test for VUS.

The Evidence-Based Network for the Interpretation of Germline Mutant Alleles (ENIGMA) consortium recognizes the importance of functional assays for classifying VUS. Further, ENIGMA has outlined a strategy for validating functional tests of BRCA2 VUS based upon IARC Class 1 (benign) and 5 (pathogenic) variants largely defined using co-segregation analyses. Such validation enables consideration of clinical data together with the results of functional tests. Useful functional tests should have a high degree of sensitivity (correct identification of pathogenic variants) and specificity (correct identification of benign variants).

A large subset of variants are missense changes. ClinVar annotates the clinical significance of human DNA sequence variants. There are ˜3,400 unique BRCA2 missense VUS currently listed in ClinVar; this is likely an underestimate as not all clinical testing laboratories contribute data to ClinVar. Many of these variants have been identified in breast-ovarian cancer families Greater than 95% of BRCA2 missense variants currently listed in ClinVar are unclassified, underscoring the abundant demand for an assay which can readily characterize their function. The disclosed methods utilize HR to predict the pathogenicity of particular VUS, as this measure has shown 100% sensitivity and specificity for pathogenic BRCA1/2 variants. The methods allow for implementation of high capacity functional assays for the many VUS in BRCA2 and related genes. Also, ˜10-15% of missense BRCA2 VUS can potentially be classified on the basis of tests for mis-splicing; following the identification of variants associated with mis-splicing using predictive algorithms, such variants will be excluded from functional tests using a codon-optimized cDNA since it cannot model mis-splicing but effects on splicing are instead confirmed using an alternative reporter assay.

The impact of previous attempts at functional assays as an alternative to classify BRCA2 VUS, which relied on heterologous expression of human BRCA2 in mouse and hamster cells, has been strongly limited by their scale. In particular, expression in murine cells using bacterial artificial chromosomes (BACs) by a process termed recombineerng is very time-consuming and therefore not readily amenable to high volume assays. Due to such limitations, together, five previous studies in rodent cells have characterized less than 5% of unique BRCA2 missense VUS using functional assays. Additionally, the human and mouse BRCA2 proteins have only 59% homology. Inexact conservation occurs to varying degrees throughout BRCA2. As a result, variants can occur in an environment with only partial homology. Importantly, inexact conservation raises concerns about both the utility and reliability of heterologous assays of BRCA2 function. Further, mouse embryonic cells and hamster fibroblasts may have limitations as a model for predicting cancer risk in humans (See, e.g., Toland A E, Andreassen P R. DNA repair-related functional assays for the classification of BRCA1 and BRCA2 variants: a critical review and needs assessment. J Med Genet. 2017; Vol. 54, pp 721-731).

To overcome the limitations of heterologous expression systems, such as the expression of human BRCA2 in rodent cells, Applicant has developed the first system for stable and efficient expression of human BRCA2 in human cells based upon vectors, for example, lentiviral vectors carrying codon-optimized full-length BRCA2. Given its unique high-volume capacity, the disclosed methods may ultimately be beneficial for assessing the risk of developing breast, ovarian and other cancers, and for guiding therapeutic decisions. Further, this system has a novel adaptability for expression of full-length human BRCA2 in any human cell type, as needed, to best address specific experimental questions. Thus, to address the potential of BRCA2 VUS for increasing the risk of developing cancer, BRCA2-deficient non-transformed cells may be utilized. Alternatively, the potential effect on the sensitivity of cancer cells to PARPi may be better determined by employing BRCA2-deficient ovarian cancer cells. This system is also adaptable to the expression of various other difficult to express proteins, including the demonstration for ATM herein, such as DNA damage response cDNAs that are very long and yield a poor mRNA quality. It will also be important to apply this system to BRCA1, since it is the most frequently mutated gene associated with hereditary breast and ovarian cancer and there are >1940 distinct BRCA1 missense VUS listed in ClinVar that remain to be tested.

The impact of the disclosed functional assays is driven by the fact they are more rapid to use and therefore have the capacity to evaluate a greater number of VUS, post-translational modifications or other introduced mutations than any previous study. This rapidity and efficiency of expression comes from the use of a cDNA carried by a lentiviral vector, or potentially other vectors, and a modular approach to accurately constructing expression constructs based upon batch synthesis and insertion into the codon-optimized cDNA utilizing unique customized restriction sites. Additionally, there is potentially improved accuracy by expressing the human protein in human cells. Further, unlike previous systems such as recombineering which could be performed only in a specific cell type, the new approach and system should permit the expression of the protein of interest in any cell line that is desired. All of the above gives the new system an unprecedented power to express proteins that are otherwise difficult to express and to resolve specific questions tailored to particular cell lines. Finally, in the case of ATM and BRCA2, the new system and approach unlocks new possibilities since, to the best of the knowledge of the applicant, there is no other system for the stable, efficient expression of these full-length proteins.

Results

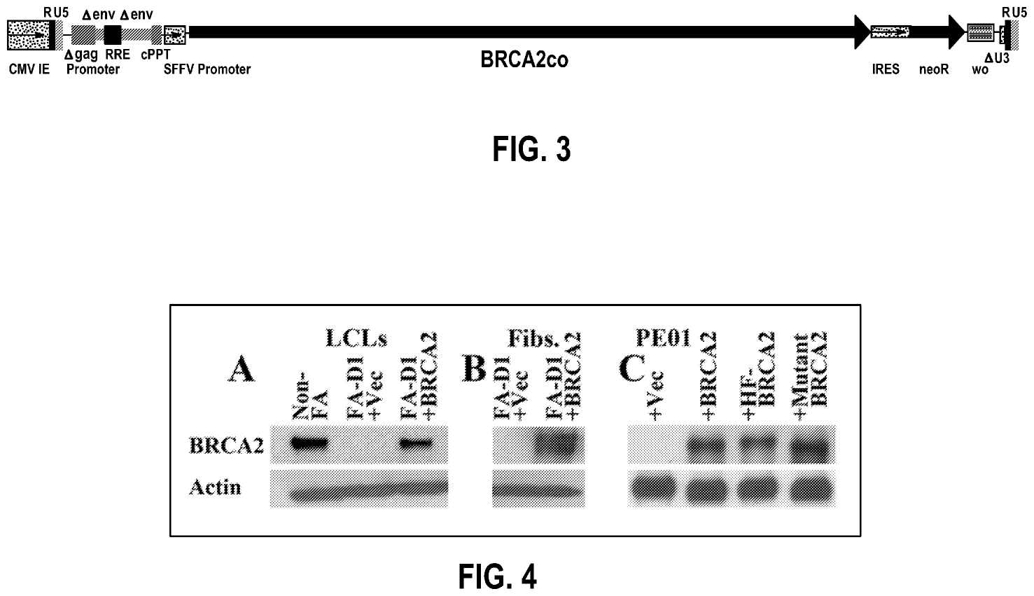

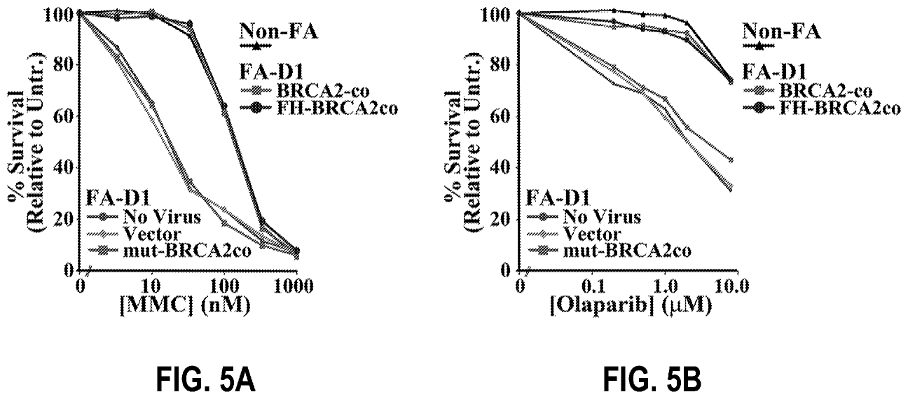

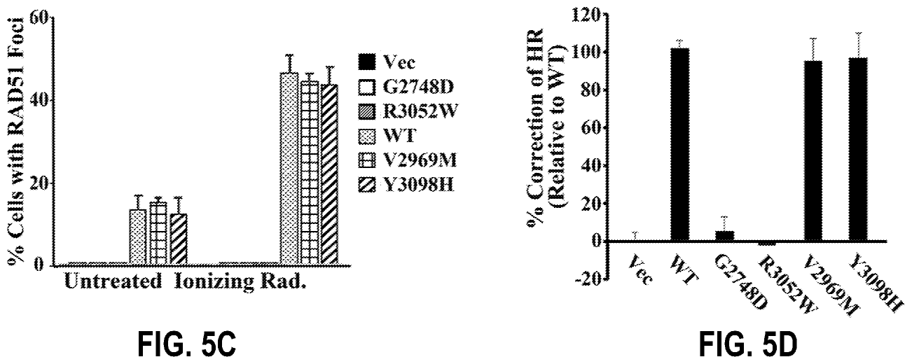

Applicant has shown that BRCA2 and its variants, as well as other breast and/or ovarian cancer genes, can successfully be expressed in human cells, and BRCA2co is able to correct defective HR and the sensitivity of BRCA2-deficient cells to PARP inhibitors (FIG. 5 B). The very large size (10,254 base pairs) and low mRNA quality of the BRCA2 cDNA have been a strong barrier to expressing the human BRCA2 protein in cells using standard plasmid transfection or viral transduction approaches. To overcome these obstacles, Applicant inserted a codon-optimized cDNA for BRCA2 into a lentiviral vector (FIG. 3). The BRCA2 cDNA utilized is engineered to contain unique restriction sites at least every 1,500 bp, and generally much more frequently. By “unique”, it is meant that the particular restriction site occurs only once in the entire BRCA2co, so depending upon whether or not particular restrictions sites occur elsewhere in the plasmid or vector backbone, fragments of BRCA2co containing variants and flanked by a unique pair of restriction sites can be synthesized for directional insertion into the entire lentiviral-BRCA2co.

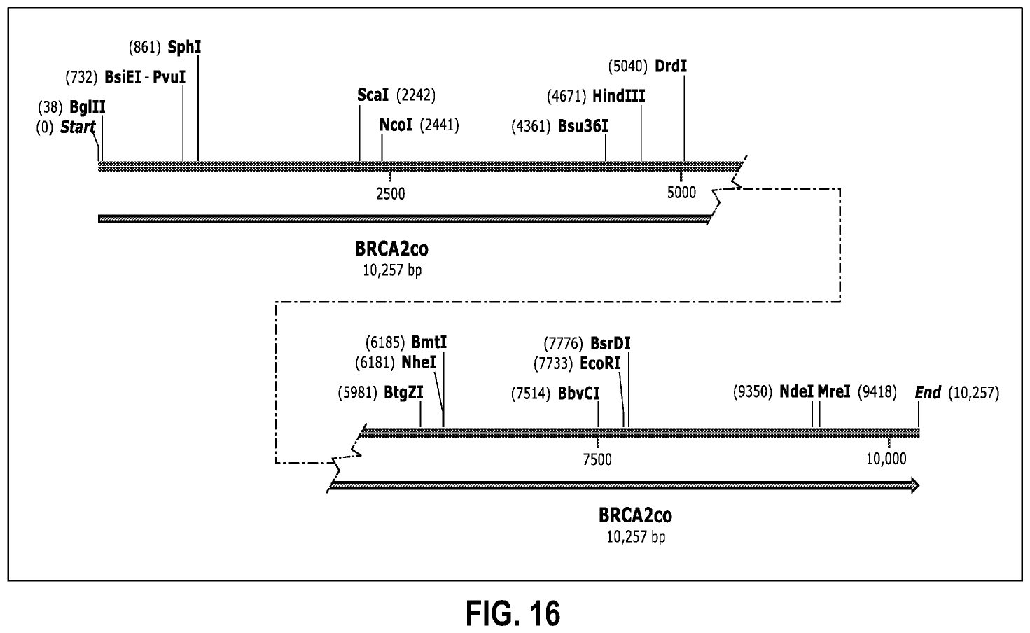

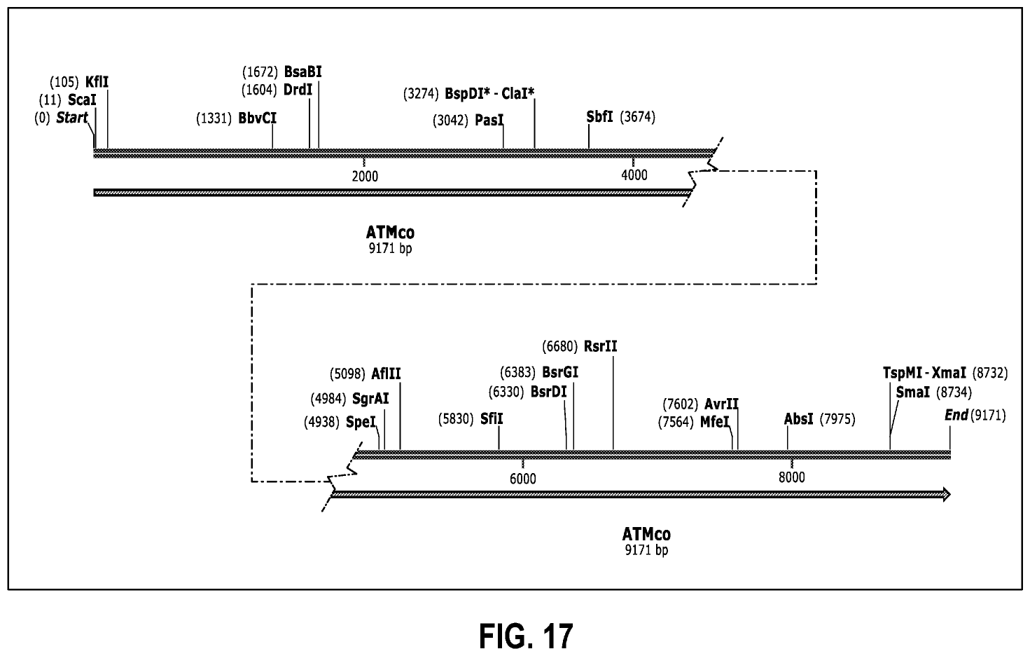

It should be noted that one of ordinary skill in the art will appreciate that there is no single codon-optimized sequence using this method. An exemplary annotated map of BRCA2co (FIG. 16) and ATMco (FIG. 17) with unique restriction sites (containing the bp position), which are specific to the codon-optimized cDNA since they do not occur at the same positions in the original, natural cDNA, is provided herein.

The disclosed system is highly adaptable for expressing human full-length BRCA2 in any human cell type. Using VSV-G as a viral envelope, human full-length BRCA2 can be expressed in more than ten human cell types, some of which are shown in FIGS. 4 and 7-8. In each case, a functional correction of the deficiency for BRCA2 using one or more DNA repair-related assays can be validated. Human BRCA2 can be expressed in BRCA2-deficient lymphoblasts (LCLs) from a FA-D1 patient at wild-type (WT) levels similar to those seen in normal control cells. BRCA2co may also be expressed in primary FA-D1 fibroblasts with a genetic deficiency for BRCA2. TERT-immortalized, non-transformed FA-D1 cells may be used to determine whether BRCA2 VUS affect DNA repair related to the role of BRCA2 in suppressing ovarian cancer.

In one exemplary embodiment, full-length BRCA2 may be efficiently expressed in human PE01 ovarian carcinoma cells that contain truncated BRCA2 but no WT protein (FIG. 4C). Also, in PE01 cells, Applicant has shown that a breast and ovarian cancer-associated missense mutation of BRCA2, c.3G>A, which removes the first methionine and thereby leads to deletion of the first 123 amino acids of BRCA2, can be virally-expressed at similar levels as WT BRCA2, either with or without a N-terminal Flag-HA epitope tag. This mutation was classified as likely pathogenic based upon a multifactorial analysis. Either WT or mutant BRCA2 can be expressed in human cells to compare their functions in the DNA damage response. Stable expression, based upon G418 selection, enables uniformity in the assays to permit the comparison of results for different variants.

The ability to stably and efficiently express BRCA2co allows for functional characterization of BRCA2 VUS in DNA repair-related assays. Cisplatin, which is a mainstay in the treatment of ovarian cancer, and mitomycin C (MMC), both induce DNA interstrand crosslinks (ICLs). WT BRCA2co conferred resistance of FA-D1 LCLs to MMC. WT BRCA2co also complemented the sensitivity of FA-D1 LCLs to the PARP inhibitor, olaparib (FIG. 5, B). Importantly, resistance to MMC or olaparib of BRCA2-deficient LCLs reconstituted with WT BRCA2co, with or without an N-terminal Flag-HA epitope tag, was indistinguishable from that of non-FA cells. Thus, codon-optimization and the epitope tag do not alter BRCA2 function in FA-D1 cells and can be utilized to reliably test BRCA2 VUS. Additionally, the breast cancer-associated c.3G>A BRCA2 missense mutation, described above, did not confer resistance to MMC or olaparib (FIG. 5, A-B). Thus, together, these assays display the ability to use the disclosed methods to functionally test wild type or mutant BRCA2.

Applicant has also shown that expression of two Class 1 benign variants, p.V2969M and p.Y3098H, and two other benign variants, p.A2717S and p.K2729N (data not shown), restore BRCA2 function in BRCA2-deficient FA-D1 fibroblasts as measured by the assembly of RAD51 nuclear foci and DSB-initiated HR. In contrast, two Class 5 pathogenic variants, p.G2748D and p.R3052W, and two other pathogenic variants, p.L2653P and p.T2722R (data not shown), are not functional. Each of these variants is in the DNA binding domain of BRCA2. Because the disclosed system readily distinguishes BRCA2 variants associated with undetectable or full loss of BRCA2 function, it is believed that it can be utilized to characterize BRCA2 VUS.

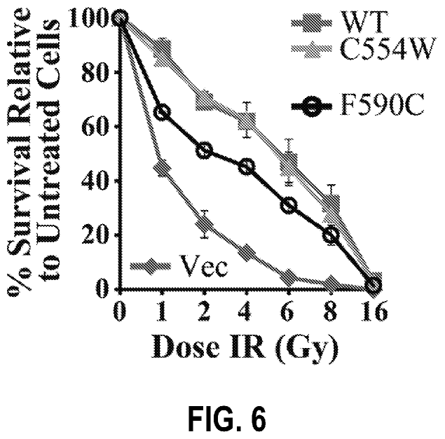

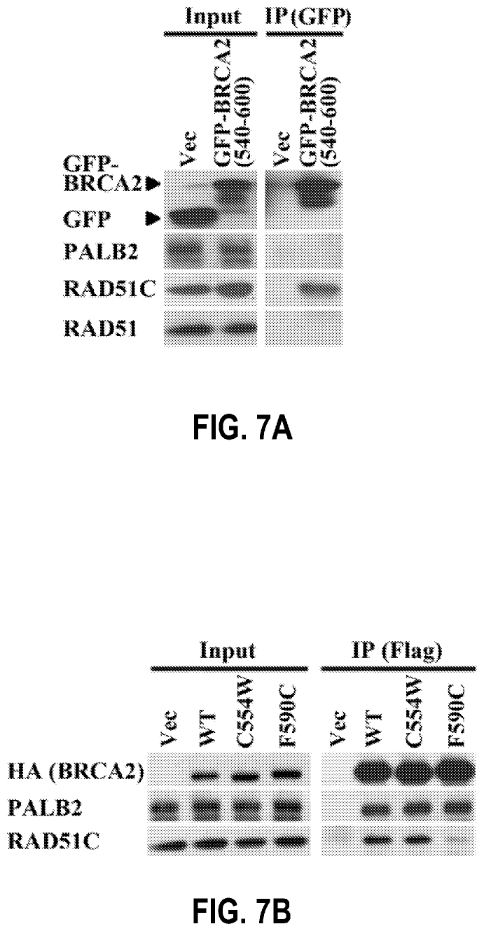

The disclosed assays may be used to detect intermediate activities associated with variants of a gene such as BRCA2. Applicant has found that amino acids (a.a.) 540-610 of BRCA2 mediate the interaction of BRCA2 with the product of another breast-ovarian cancer susceptibility gene, RAD51C (FIG. 7). Two BRCA2 VUS, p.C554W and p.F590C, lie within the RAD51C-binding domain have been tested utilizing the newly developed expression system. When expressed in full-length BRCA2, the p.F590C variant conferred resistance to ionizing radiation (IR) that was intermediate to that found in FA-D1 cells expressing WT BRCA2co or which contained the empty vector (FIG. 6). These results demonstrate a functional interaction between the products of two ovarian cancer tumor suppressor genes. In contrast, results obtained with C554W, previously classified as benign based upon a multifactorial analysis, were not significantly different than for cells corrected with WT BRCA2co. Along with FIG. 5, these results indicate that the disclosed assays are sufficiently robust to distinguish BRCA2 VUS associated with no, partial or full loss of BRCA2 function.

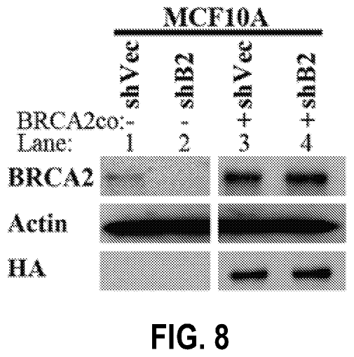

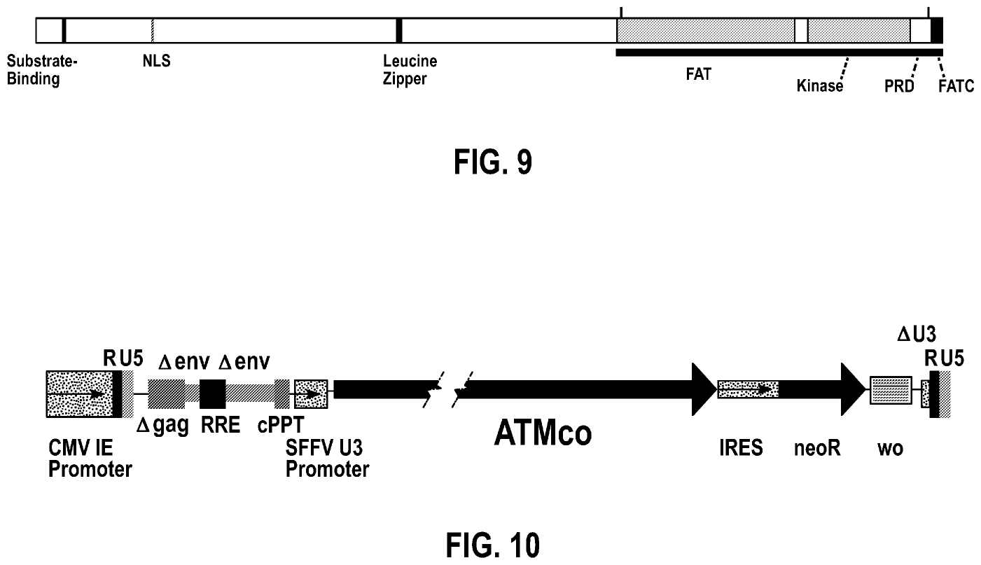

Another cell line in which BRCA2co has been expressed is MCF10A non-transformed human mammary epithelial cells either with or without expression of a shRNA that depletes endogenous BRCA2 (FIG. 8). Codon-optimization renders the cDNA resistant to the shBRCA2 utilized. Additionally, this system could be utilized to test the effects of BRCA2 VUS on cancer risk in non-transformed mammary epithelial cells as a pre-neoplastic model for breast cancer. Also, this again demonstrates the versatility of the system to express BRCA2co in various types of human cells. This codon-optimization based system should also be adaptable to related DNA damage response proteins, many of them very large, that have been otherwise difficult to express, including ATR, BRCA1, CHEK1, FANCM and PRKDC (DNA-PK catalytic subunit).

### REFERENCES

Weissman S M, Weiss S M, Newlin A C. Genetic testing by cancer site: ovary. Cancer J. 2012; 18(4):320-327.
Norquist B M, Harrell M I, Brady M F, et al. Inherited Mutations in Women With Ovarian Carcinoma. JAMA Oncol. 2016; 2(4):482-490.
Walsh T, Casadei S, Lee M K, et al. Mutations in 12 genes for inherited ovarian, fallopian tube, and peritoneal carcinoma identified by massively parallel sequencing. Proc Natl Acad Sci USA. 2011; 108(44):18032-18037.
Wu K, Hinson S R, Ohashi A, et al. Functional evaluation and cancer risk assessment of BRCA2 unclassified variants. Cancer Res. 2005; 65(2):417-426.
Kuznetsov S G, Liu P, Sharan S K. Mouse embryonic stem cell-based functional assay to evaluate mutations in BRCA2. Nat Med. 2008; 14(8):875-881.
Biswas K, Das R, Eggington J M, et al. Functional evaluation of BRCA2 variants mapping to the PALB2-binding and C-terminal DNA-binding domains using a mouse ES cell-based assay. Hum Mol Genet. 2012; 21(18):3993-4006.
Guidugli L, Pankratz V S, Singh N, et al. A classification model for BRCA2 DNA binding domain missense variants based on homology-directed repair activity. Cancer Res. 2013; 73(1):265-275.
Friebel T M, Domchek S M, Rebbeck T R. Modifiers of cancer risk in BRCA1 and BRCA2 mutation carriers: systematic review and meta-analysis. J Natl Cancer Inst. 2014; 106(6):dju091.
Shannon K M, Chittenden A. Genetic testing by cancer site: breast. Cancer J. 2012; 18(4):310-319.
Chen S, Parmigiani G. Meta-analysis of BRCA1 and BRCA2 penetrance. J Clin Oncol. 2007; 25(11):1329-1333.
Pennington K P, Walsh T, Harrell M I, et al. Germline and somatic mutations in homologous recombination genes predict platinum response and survival in ovarian, fallopian tube, and peritoneal carcinomas. Clin Cancer Res. 2014; 20(3):764-775.
Chen F M, Hou M F, Chang M Y, et al. High frequency of somatic missense mutation of BRCA2 in female breast cancer from Taiwan. Cancer Lett. 2005; 220(2):177-184.
Dann R B, DeLoia J A, Timms K M, et al. BRCA1/2 mutations and expression: response to platinum chemotherapy in patients with advanced stage epithelial ovarian cancer. Gynecol Oncol. 2012; 125(3):677-682.
Mafficini A, Simbolo M, Parisi A, et al. BRCA somatic and germline mutation detection in paraffin embedded ovarian cancers by next-generation sequencing. Oncotarget. 2016; 7(2):1076-1083.
Howlett N G, Taniguchi T, Olson S, et al. Biallelic inactivation of BRCA2 in Fanconi anemia. Science. 2002; 297(5581):606-609.
Sawyer S L, Tian L, Kahkonen M, et al. Biallelic mutations in BRCA1 cause a new Fanconi anemia subtype. Cancer Discov. 2015; 5(2):135-142.
Venkitaraman A R. Functions of BRCA1 and BRCA2 in the biological response to DNA damage. J Cell Sci. 2001; 114(Pt 20):3591-3598.
Moynahan M E, Jasin M. Mitotic homologous recombination maintains genomic stability and suppresses tumorigenesis. Nat Rev Mol Cell Biol. 2010; 11(3):196-207.
Buisson R, Dion-Cote A M, Coulombe Y, et al. Cooperation of breast cancer proteins PALB2 and piccolo BRCA2 in stimulating homologous recombination. Nat Struct Mol Biol. 2010; 17(10):1247-1254.
Sung P, Robberson D L. DNA strand exchange mediated by a RAD51-ssDNA nucleoprotein filament with polarity opposite to that of RecA. Cell. 1995; 82(3):453-461.
Baumann P, Benson F E, West S C. Human Rad51 protein promotes ATP-dependent homologous pairing and strand transfer reactions in vitro. Cell. 1996; 87(4):757-766.
Xia B, Sheng Q, Nakanishi K, et al. Control of BRCA2 cellular and clinical functions by a nuclear partner, PALB2. Mol Cell. 2006; 22(6):719-729.
Zhang F, Fan Q, Ren K, Andreassen P R. PALB2 functionally connects the breast cancer susceptibility proteins BRCA1 and BRCA2. Mol Cancer Res. 2009; 7(7):1110-1118.
Zhang F, Ma J, Wu J, et al. PALB2 links BRCA1 and BRCA2 in the DNA-damage response. Curr Biol. 2009; 19(6):524-529.
Sy S M, Huen M S, Chen J. PALB2 is an integral component of the BRCA complex required for homologous recombination repair. Proc Natl Acad Sci USA. 2009; 106(17):7155-7160.
Park J Y, Singh T R, Nassar N, et al. Breast cancer-associated missense mutants of the PALB2 WD40 domain, which directly binds RAD51C, RAD51 and BRCA2, disrupt DNA repair. Oncogene. 2014; 33(40):4803-4812.
Kumaraswamy E, Shiekhattar R. Activation of BRCA1/BRCA2-associated helicase BACH1 is required for timely progression through S phase. Mol Cell Biol. 2007; 27(19): 6733-6741.
Bignell G, Micklem G, Stratton M R, Ashworth A, Wooster R. The BRC repeats are conserved in mammalian BRCA2 proteins. Hum Mol Genet. 1997; 6(1):53-58.
Chen P L, Chen C F, Chen Y, Xiao J, Sharp Z D, Lee W H. The BRC repeats in BRCA2 are critical for RAD51 binding and resistance to methyl methanesulfonate treatment. Proc Natl Acad Sci USA. 1998; 95(9):5287-5292.
Carreira A, Kowalczykowski S C. Two classes of BRC repeats in BRCA2 promote RAD51 nucleoprotein filament function by distinct mechanisms. Proc Natl Acad Sci USA. 2011; 108(26):10448-10453.
Yang H, Jeffrey P D, Miller J, et al. BRCA2 function in DNA binding and recombination from a BRCA2-DSS1-ssDNA structure. Science. 2002; 297(5588):1837-1848.
von Nicolai C, Ehlen A, Martin C, Zhang X, Carreira A. A second DNA binding site in human BRCA2 promotes homologous recombination. Nat Commun. 2016; 7:12813.
Davies O R, Pellegrini L. Interaction with the BRCA2 C terminus protects RAD51-DNA filaments from disassembly by BRC repeats. Nat Struct Mol Biol. 2007; 14(6):475-483.
Frank T S, Deffenbaugh A M, Reid J E, et al. Clinical characteristics of individuals with germline mutations in BRCA1 and BRCA2: analysis of 10,000 individuals. J Clin Oncol. 2002; 20(6):1480-1490.
Michl J, Zimmer J, Tarsounas M. Interplay between Fanconi anemia and homologous recombination pathways in genome integrity. EMBO J. 2016; 35(9):909-923.
Prakash R, Zhang Y, Feng W, Jasin M. Homologous recombination and human health: the roles of BRCA1, BRCA2, and associated proteins. Cold Spring Harb Perspect Biol. 2015; 7(4):a016600.
Zhang F, Bick G, Park J Y, Andreassen P R. MDC1 and RNF8 function in a pathway that directs BRCA1-dependent localization of PALB2 required for homologous recombination. J Cell Sci. 2012; 125(Pt 24):6049-6057.
Schild D, Lio Y C, Collins D W, Tsomondo T, Chen D J. Evidence for simultaneous protein interactions between human Rad51 paralogs. J Biol Chem. 2000; 275(22):16443-16449.
Miesfeldt S, Lamb A, Duarte C. Management of genetic syndromes predisposing to gynecologic cancers. Curr Treat Options Oncol. 2013; 14(1):34-50.
Rebbeck T R, Kauff N D, Domchek S M. Meta-analysis of risk reduction estimates associated with risk-reducing salpingo-oophorectomy in BRCA1 or BRCA2 mutation carriers. J Natl Cancer Inst. 2009; 101(2):80-87.
Rivera C M, Grossardt B R, Rhodes D J, et al. Increased cardiovascular mortality after early bilateral oophorectomy. Menopause. 2009; 16(1):15-23.
McLemore M R, Miaskowski C, Aouizerat B E, Chen L M, Dodd M J. Epidemiological and genetic factors associated with ovarian cancer. Cancer Nurs. 2009; 32(4):281-288.
Guindalini R S, Song A, Fackenthal J D, Olopade O I, Huo D. Genetic anticipation in BRCA1/BRCA2 families after controlling for ascertainment bias and cohort effect. Cancer. 2016; 122(12):1913-1920.
Maxwell K N, Domchek S M. Cancer treatment according to BRCA1 and BRCA2 mutations. Nat Rev Clin Oncol. 2012; 9(9):520-528.
Alsop K, Fereday S, Meldrum C, et al. BRCA mutation frequency and patterns of treatment response in BRCA mutation-positive women with ovarian cancer: a report from the Australian Ovarian Cancer Study Group. J Clin Oncol. 2012; 30(21):2654-2663.
Konstantinopoulos P A, Spentzos D, Karlan B Y, et al. Gene expression profile of BRCAness that correlates with responsiveness to chemotherapy and with outcome in patients with epithelial ovarian cancer. J Clin Oncol. 2010; 28(22):3555-3561.
Bryant H E, Schultz N, Thomas H D, et al. Specific killing of BRCA2-deficient tumours with inhibitors of poly(ADP-ribose) polymerase. Nature. 2005; 434(7035):913-917.
Farmer H, McCabe N, Lord C J, et al. Targeting the DNA repair defect in BRCA mutant cells as a therapeutic strategy. Nature. 2005; 434(7035):917-921.
Mirza M R, Monk B J, Herrstedt J, et al. Niraparib Maintenance Therapy in Platinum-Sensitive, Recurrent Ovarian Cancer. N Engl J Med. 2016; 375(22):2154-2164.
Kondrashova O, Nguyen M, Shield-Artin K, et al. Secondary Somatic Mutations Restoring RAD51C and RAD51D Associated with Acquired Resistance to the PARP Inhibitor Rucaparib in High-Grade Ovarian Carcinoma. Cancer Discov. 2017; 7(9):984-998.
Quigley D, Alumkal J J, Wyatt A W, et al. Analysis of Circulating Cell-Free DNA Identifies Multiclonal Heterogeneity of BRCA2 Reversion Mutations Associated with Resistance to PARP Inhibitors. Cancer Discov. 2017; 7(9):999-1005.
Swisher E M, Lin K K, Oza A M, et al. Rucaparib in relapsed, platinum-sensitive high-grade ovarian carcinoma (ARIEL2 Part 1): an international, multicentre, open-label, phase 2 trial. Lancet Oncol. 2017; 18(1):75-87.
Walsh C S. Two decades beyond BRCA1/2: Homologous recombination, hereditary cancer risk and a target for ovarian cancer therapy. Gynecol Oncol. 2015; 137(2):343-350.
Suh D H, Lee K H, Kim K, Kang S, Kim J W. Major clinical research advances in gynecologic cancer in 2014. J Gynecol Oncol. 2015; 26(2):156-167.
Rucaparib Approved for Ovarian Cancer. Cancer Discov. 2017; 7(2):120-121.
Friedlander M, Banerjee S, Mileshkin L, Scott C, Shannon C, Goh J. Practical guidance on the use of olaparib capsules as maintenance therapy for women with BRCA mutations and platinum-sensitive recurrent ovarian cancer. Asia Pac J Clin Oncol. 2016; 12(4):323-331.
Ledermann J A, Harter P, Gourley C, et al. Overall survival in patients with platinum-sensitive recurrent serous ovarian cancer receiving olaparib maintenance monotherapy: an updated analysis from a randomised, placebo-controlled, double-blind, phase 2 trial. Lancet Oncol. 2016; 17(11):1579-1589.
Spurdle A B, Healey S, Devereau A, et al. ENIGMA—evidence-based network for the interpretation of germline mutant alleles: an international initiative to evaluate risk and clinical significance associated with sequence variation in BRCA1 and BRCA2 genes. Hum Mutat. 2012; 33(1):2-7.
Guidugli L, Carreira A, Caputo S M, et al. Functional assays for analysis of variants of uncertain significance in BRCA2. Hum Mutat. 2014; 35(2):151-164.
Lindor N M, Guidugli L, Wang X, et al. A review of a multifactorial probability-based model for classification of BRCA1 and BRCA2 variants of uncertain significance (VUS). Hum Mutat. 2012; 33(1):8-21.
Eggington J M, Bowles K R, Moyes K, et al. A comprehensive laboratory-based program for classification of variants of uncertain significance in hereditary cancer genes. Clin Genet. 2014; 86(3):229-237.
Plon S E, Eccles D M, Easton D, et al. Sequence variant classification and reporting: recommendations for improving the interpretation of cancer susceptibility genetic test results. Hum Mutat. 2008; 29(11):1282-1291.
Polak P, Kim J, Braunstein L Z, et al. A mutational signature reveals alterations underlying deficient homologous recombination repair in breast cancer. Nat Genet. 2017; (Epub August 21), doi:10.1038/ng.3934.
Akbari M R, Zhang S, Fan I, et al. Clinical impact of unclassified variants of the BRCA1 and BRCA2 genes. J Med Genet. 2011; 48(11):783-786.
Landrum M J, Lee J M, Benson M, et al. ClinVar: public archive of interpretations of clinically relevant variants. Nucleic Acids Res. 2016; 44(D1):D862-868.
Toland A E, Andreassen P R. DNA repair-related functional assays for the classification of BRCA1 and BRCA2 variants: a critical review and needs assessment. J Med Genet. 2017; (Epub September 2), doi:10.1136/jmedgenet-2017-104707.
Connor F, Smith A, Wooster R, et al. Cloning, chromosomal mapping and expression pattern of the mouse Brca2 gene. Hum Mol Genet. 1997; 6(2):291-300.
McAllister K A, Haugen-Strano A, Hagevik S, et al. Characterization of the rat and mouse homologues of the BRCA2 breast cancer susceptibility gene. Cancer Res. 1997; 57(15):3121-3125.
Mechold U, Gilbert C, Ogryzko V. Codon optimization of the BirA enzyme gene leads to higher expression and an improved efficiency of biotinylation of target proteins in mammalian cells. J Biotechnol. 2005; 116(3):245-249.
Carton J M, Sauerwald T, Hawley-Nelson P, et al. Codon engineering for improved antibody expression in mammalian cells. Protein Expr Purif. 2007; 55(2):279-286.
Chang S, Biswas K, Martin B K, Stauffer S, Sharan S K. Expression of human BRCA1 variants in mouse E S cells allows functional analysis of BRCA1 mutations. J Clin Invest. 2009; 119(10):3160-3171.
Ransburgh D J, Chiba N, Ishioka C, Toland A E, Parvin J D. Identification of breast tumor mutations in BRCA1 that abolish its function in homologous DNA recombination. Cancer Res. 2010; 70(3):988-995.
Towler W I, Zhang J, Ransburgh D J, et al. Analysis of BRCA1 variants in double-strand break repair by homologous recombination and single-strand annealing. Hum Mutat. 2013; 34(3):439-445.
Lee C, Banerjee T, Gillespie J, et al. Functional Analysis of BARD1 Missense Variants in Homology-Directed Repair of DNA Double Strand Breaks. Hum Mutat. 2015; 36(12):1205-1214.
Rasmussen U, Heinen C D, Royer-Pokora B, et al. Pathological assessment of mismatch repair gene variants in Lynch syndrome: past, present, and future. Hum Mutat. 2012; 33(12):1617-1625.
Sakai W, Swisher E M, Jacquemont C, et al. Functional restoration of BRCA2 protein by secondary BRCA2 mutations in BRCA2-mutated ovarian carcinoma. Cancer Res. 2009; 69(16):6381-6386.
Thomassen M, Blanco A, Montagna M, et al. Characterization of BRCA1 and BRCA2 splicing variants: a collaborative report by ENIGMA consortium members. Breast Cancer Res Treat. 2012; 132(3):1009-1023.
Garcia-Saenz J A, Custodio A, Casado A, et al. Platinum-based adjuvant chemotherapy on moderate- and high-risk stage I and II epithelian ovarian cancer patients. Long-term single institution experience and literature review. Clin Transl Oncol. 2011; 13(2):121-132.
Thigpen T, Vance R, Puneky L, Khansur T. Chemotherapy in advanced ovarian carcinoma: current standards of care based on randomized trials. Gynecol Oncol. 1994; 55(3 Pt 2):597-107.
Andreassen P R, Ren K. Fanconi anemia proteins, DNA interstrand crosslink repair pathways, and cancer therapy. Curr Cancer Drug Targets. 2009; 9(1):101-117.
Meindl A, Hellebrand H, Wiek C, et al. Germline mutations in breast and ovarian cancer pedigrees establish RAD51C as a human cancer susceptibility gene. Nat Genet. 2010; 42(5):410-414.
Vuorela M, Pylkas K, Hartikainen J M, et al. Further evidence for the contribution of the RAD51C gene in hereditary breast and ovarian cancer susceptibility. Breast Cancer Res Treat. 2011; 130(3):1003-1010.
Easton D F, Deffenbaugh A M, Pruss D, et al. A systematic genetic assessment of 1,433 sequence variants of unknown clinical significance in the BRCA1 and BRCA2 breast cancer-predisposition genes. Am J Hum Genet. 2007; 81(5):873-883.
Li J, Meeks H, Feng B J, et al. Targeted massively parallel sequencing of a panel of putative breast cancer susceptibility genes in a large cohort of multiple-case breast and ovarian cancer families J Med Genet. 2016; 53(1):34-42.
Zhang F, Fan Q, Ren K, Auerbach A D, Andreassen P R. FANCJ/BRIP1 recruitment and regulation of FANCD2 in DNA damage responses. Chromosoma. 2010; 119(6):637-649.
Li N F, Broad S, Lu Y J, et al. Human ovarian surface epithelial cells immortalized with hTERT maintain functional pRb and p53 expression. Cell Prolif. 2007; 40(5):780-794.
Jiang X R, Jimenez G, Chang E, et al. Telomerase expression in human somatic cells does not induce changes associated with a transformed phenotype. Nat Genet. 1999; 21(1):111-114.
Morales C P, Holt S E, Ouellette M, et al. Absence of cancer-associated changes in human fibroblasts immortalized with telomerase. Nat Genet. 1999; 21(1):115-118.
Ouellette M M, McDaniel L D, Wright W E, Shay J W, Schultz R A. The establishment of telomerase-immortalized cell lines representing human chromosome instability syndromes. Hum Mol Genet. 2000; 9(3):403-411.
Bouwman P, van der Gulden H, van der Heijden I, et al. A high-throughput functional complementation assay for classification of BRCA1 missense variants. Cancer Discov. 2013; 3(10):1142-1155.
Rebbeck T R, Mitra N, Wan F, et al. Association of type and location of BRCA1 and BRCA2 mutations with risk of breast and ovarian cancer. JAMA. 2015; 313(13):1347-1361.
Vallee M P, Di Sera T L, Nix D A, et al. Adding In Silico Assessment of Potential Splice Aberration to the Integrated Evaluation of BRCA Gene Unclassified Variants. Hum Mutat. 2016; 37(7):627-639.
Hartmann L, Neveling K, Borkens S, et al. Correct mRNA processing at a mutant T T splice donor in FANCC ameliorates the clinical phenotype in patients and is enhanced by delivery of suppressor U1 snRNAs. Am J Hum Genet. 2010; 87(4):480-493.
Wang X, Andreassen P R, D'Andrea A D. Functional interaction of monoubiquitinated FANCD2 and BRCA2/FANCD1 in chromatin. Mol Cell Biol. 2004; 24(13):5850-5862.
Hayakawa T, Zhang F, Hayakawa N, et al. MRG15 binds directly to PALB2 and stimulates homology-directed repair of chromosomal breaks. J Cell Sci. 2010; 123(Pt 7):1124-1130.
Fan Q, Zhang F, Barrett B, Ren K, Andreassen P R. A role for monoubiquitinated FANCD2 at telomeres in ALT cells. Nucleic Acids Res. 2009; 37(6):1740-1754.
Taniguchi T, Garcia-Higuera I, Xu B, et al. Convergence of the fanconi anemia and ataxia telangiectasia signaling pathways. Cell. 2002; 109(4):459-472.
Pierce A J, Johnson R D, Thompson L H, Jasin M. XRCC3 promotes homology-directed repair of DNA damage in mammalian cells. Genes Dev. 1999; 13(20):2633-2638.
Spearman A D, Sweet K, Zhou X P, McLennan J, Couch F J, Toland A E. Clinically applicable models to characterize BRCA1 and BRCA2 variants of uncertain significance. J Clin Oncol. 2008; 26(33):5393-5400.
Tavtigian S V, Byrnes G B, Goldgar D E, Thomas A. Classification of rare missense substitutions, using risk surfaces, with genetic- and molecular-epidemiology applications. Hum Mutat. 2008; 29(11):1342-1354.
Spurdle A B, Couch F J, Parsons M T, et al. Refined histopathological predictors of BRCA1 and BRCA2 mutation status: a large-scale analysis of breast cancer characteristics from the BCAC, CIMBA, and ENIGMA consortia. Breast Cancer Res. 2014; 16(6):3419.
Mohammadi L, Vreeswijk M P, Oldenburg R, et al. A simple method for co-segregation analysis to evaluate the pathogenicity of unclassified variants; BRCA1 and BRCA2 as an example. BMC Cancer. 2009; 9:211.
Gomez Garcia E B, Oosterwijk J C, Timmermans M, et al. A method to assess the clinical significance of unclassified variants in the BRCA1 and BRCA2 genes based on cancer family history. Breast Cancer Res. 2009; 11(1):R8.
Shimelis H, Mesman R L S, Von Nicolai C, et al. BRCA2 Hypomorphic Missense Variants Confer Moderate Risks of Breast Cancer. Cancer Res. 2017; 77(11):2789-2799.
Drost R, Bouwman P, Rottenberg S, et al. BRCA1 RING function is essential for tumor suppression but dispensable for therapy resistance. Cancer Cell. 2011; 20(6):797-809.
Saeki H, Siaud N, Christ N, et al. Suppression of the DNA repair defects of BRCA2-deficient cells with heterologous protein fusions. Proc Natl Acad Sci USA. 2006; 103(23):8768-8773.
Tewari K S, Eskander R N, Monk B J. Development of Olaparib for BRCA-Deficient Recurrent Epithelial Ovarian Cancer. Clin Cancer Res. 2015; 21(17):3829-3835.
Maxwell K N, Wubbenhorst B, Wenz B M, et al. BRCA locus-specific loss of heterozygosity in germline BRCA1 and BRCA2 carriers. Nat Commun. 2017; 8(1):319.
Litman R, Peng M, Jin Z, et al. BACH1 is critical for homologous recombination and appears to be the Fanconi anemia gene product FANCJ. Cancer Cell. 2005; 8(3):255-265.
Somyajit K, Subramanya S, Nagaraju G. Distinct roles of FANCO/RAD51C protein in DNA damage signaling and repair: implications for Fanconi anemia and breast cancer susceptibility. J Biol Chem. 2012; 287(5):3366-3380.
Takata M, Sasaki M S, Tachiiri S, et al. Chromosome instability and defective recombinational repair in knockout mutants of the five Rad51 paralogs. Mol Cell Biol. 2001; 21(8):2858-2866.
Lombardi A J, Hoskins E E, Foglesong G D, et al. Acquisition of Relative Interstrand Crosslinker Resistance and PARP Inhibitor Sensitivity in Fanconi Anemia Head and Neck Cancers. Clin Cancer Res. 2015; 21(8):1962-1972.
Ghosh S, Sur S, Yerram S R, et al. Hypersensitivities for acetaldehyde and other agents among cancer cells null for clinically relevant Fanconi anemia genes. Am J Pathol. 2014; 184(1):260-270.
Park J Y, Virts E L, Jankowska A, et al. Complementation of hypersensitivity to DNA interstrand crosslinking agents demonstrates that XRCC2 is a Fanconi anaemia gene. J Med Genet. 2016; 53(10):672-680.
Hinz J M, Tebbs R S, Wilson P F, et al. Repression of mutagenesis by Rad51D-mediated homologous recombination. Nucleic Acids Res. 2006; 34(5):1358-1368.
Wiese C, Hinz J M, Tebbs R S, et al. Disparate requirements for the Walker A and B ATPase motifs of human RAD51D in homologous recombination. Nucleic Acids Res. 2006; 34(9):2833-2843.
Siegel R L, Miller K D, Jemal A. Cancer statistics, 2015. C A Cancer J Clin. 2015; 65(1):5-29.
Torre L A, Bray F, Siegel R L, Ferlay J, Lortet-Tieulent J, Jemal A. Global cancer statistics, 2012. C A Cancer J Clin. 2015; 65(2):87-108.
Santarpia L, Bottai G, Kelly C M, Gyorffy B, Szekely B, Pusztai L. Deciphering and Targeting Oncogenic Mutations and Pathways in Breast Cancer. Oncologist. 2016; 21(9): 1063-1078.
Ziats M N, Rennert O M. The Evolving Diagnostic and Genetic Landscapes of Autism Spectrum Disorder. Front Genet. 2016; 7:65.
DiStefano M T, Hemphill S E, Cushman B J, et al. Curating clinically relevant transcripts for the interpretation of sequence variants. J Mol Diagn. 2018.
Rupp S, Felimban M, Schanzer A, et al. Genetic basis of hypertrophic cardiomyopathy in children. Clin Res Cardiol. 2018.
Xu Y, Liu X, Shen J, et al. The Whole Exome Sequencing Clarifies the Genotype-Phenotype Correlations in Patients with Early-Onset Dementia. Aging Dis. 2018; 9(4):696-705.
Clarke A J, Wallgren-Pettersson C. Ethics in genetic counselling. J Community Genet. 2018.
Weile J, Roth F P. Multiplexed assays of variant effects contribute to a growing genotype-phenotype atlas. Hum Genet. 2018.
Shirts B H, Pritchard C C, Walsh T Family-Specific Variants and the Limits of Human Genetics. Trends Mol Med. 2016; 22(11):925-934.
Shah P D, Nathanson K L. Application of Panel-Based Tests for Inherited Risk of Cancer. Annu Rev Genomics Hum Genet. 2017; 18:201-227.
Cheong P L, Caramins M. Approaches for classifying DNA variants found by Sanger sequencing in a medical genetics laboratory. Methods Mol Biol. 2014; 1168:227-250.
Turnbull C, Sud A, Houlston R S. Cancer genetics, precision prevention and a call to action. Nat Genet. 2018; 50(9):1212-1218.
Shiloh Y, Ziv Y. The ATM protein kinase: regulating the cellular response to genotoxic stress, and more. Nat Rev Mol Cell Biol. 2013; 14(4):197-210.
Guleria A, Chandna S. ATM kinase: Much more than a DNA damage responsive protein. DNA Repair (Amst). 2016; 39:1-20.
Rothblum-Oviatt C, Wright J, Lefton-Greif M A, McGrath-Morrow S A, Crawford T O, Lederman H M. Ataxia telangiectasia: a review. Orphanet J Rare Dis. 2016; 11(1):159.
Boder E, Sedgwick R P. Ataxia-telangiectasia; a familial syndrome of progressive cerebellar ataxia, oculocutaneous telangiectasia and frequent pulmonary infection. Pediatrics. 1958; 21(4):526-554.
McFarlin D E, Strober W, Waldmann T A. Ataxia-telangiectasia. Medicine (Baltimore). 1972; 51(4):281-314.
Savitsky K, Bar-Shira A, Gilad S, et al. A single ataxia telangiectasia gene with a product similar to PI-3 kinase. Science. 1995; 268(5218):1749-1753.
Ahmed M, Rahman N ATM and breast cancer susceptibility. Oncogene. 2006; 25(43):5906-5911.
Roberts N J, Norris A L, Petersen G M, et al. Whole Genome Sequencing Defines the Genetic Heterogeneity of Familial Pancreatic Cancer. Cancer Discov. 2016; 6(2): 166-175.
Benito-Vicente A, Uribe K B, Jebari S, Galicia-Garcia U, Ostolaza H, Martin C. Validation of LDLr Activity as a Tool to Improve Genetic Diagnosis of Familial Hypercholesterolemia: A Retrospective on Functional Characterization of LDLr Variants. Int J Mol Sci. 2018; 19(6).
Hebert E, Borngraber F, Schmidt A, et al. Functional Characterization of Rare RAB12 Variants and Their Role in Musician's and Other Dystonias. Genes (Basel). 2017; 8(10).
Toland A E, Andreassen P R. DNA repair-related functional assays for the classification of BRCA1 and BRCA2 variants: a critical review and needs assessment. J Med Genet. 2017; 54(11):721-731.
Hughes R A, Miklos A E, Ellington A D. Gene synthesis: methods and applications. Methods Enzymol. 2011; 498:277-309.
Ma S, Tang N, Tian J. DNA synthesis, assembly and applications in synthetic biology. Curr Opin Chem Biol. 2012; 16(3-4):260-267.
Cremona C A, Behrens A. ATM signalling and cancer. Oncogene. 2014; 33(26):3351-3360.
Lee J H, Paull T T. ATM activation by DNA double-strand breaks through the Mre11-Rad50-Nbs1 complex. Science. 2005; 308(5721):551-554.
Bahcall O G. Genetic testing. ACMG guides on the interpretation of sequence variants. Nat Rev Genet. 2015; 16(5):256-257.
Richards S, Aziz N, Bale S, et al. Standards and guidelines for the interpretation of sequence variants: a joint consensus recommendation of the American College of Medical Genetics and Genomics and the Association for Molecular Pathology. Genet Med. 2015; 17(5):405-424.
Mehta A, Haber J E. Sources of DNA double-strand breaks and models of recombinational DNA repair. Cold Spring Harb Perspect Biol. 2014; 6(9):a016428.
Jackson S P. Sensing and repairing DNA double-strand breaks. Carcinogenesis. 2002; 23(5):687-696.
Cunlift P N, Mann J R, Cameron A H, Roberts K D, Ward H N. Radiosensitivity in ataxia-telangiectasia. Br J Radiol. 1975; 48(569):374-376.
Helgason H, Rafnar T, Olafsdottir H S, et al. Loss-of-function variants in ATM confer risk of gastric cancer. Nat Genet. 2015; 47(8):906-910.
Greenman C, Stephens P, Smith R, et al. Patterns of somatic mutation in human cancer genomes. Nature. 2007; 446(7132):153-158.
Choi M, Kipps T, Kurzrock R. ATM Mutations in Cancer: Therapeutic Implications. Mol Cancer Ther. 2016; 15(8):1781-1791.
Matsuoka S, Ballif B A, Smogorzewska A, et al. ATM and ATR substrate analysis reveals extensive protein networks responsive to DNA damage. Science. 2007; 316(5828):1160-1166.
Bensimon A, Schmidt A, Ziv Y, et al. ATM-dependent and -independent dynamics of the nuclear phosphoproteome after DNA damage. Sci Signal. 2010; 3(151):rs3.
Melchionna R, Chen X B, Blasina A, McGowan C H. Threonine 68 is required for radiation-induced phosphorylation and activation of Cds1. Nat Cell Biol. 2000; 2(10):762-765.
Siliciano J D, Canman C E, Taya Y, Sakaguchi K, Appella E, Kastan M B. DNA damage induces phosphorylation of the amino terminus of p53. Genes Dev. 1997; 11(24):3471-3481.
Cortez D, Wang Y, Qin J, Elledge S J. Requirement of ATM-dependent phosphorylation of brca1 in the DNA damage response to double-strand breaks. Science. 1999; 286(5442):1162-1166.
Lim D S, Kim S T, Xu B, et al. ATM phosphorylates p95/nbs1 in an S-phase checkpoint pathway. Nature. 2000; 404(6778):613-617.
Wu X, Ranganathan V, Weisman D S, et al. ATM phosphorylation of Nijmegen breakage syndrome protein is required in a DNA damage response. Nature. 2000; 405(6785):477-482.
Morgan S E, Lovly C, Pandita T K, Shiloh Y, Kastan M B. Fragments of ATM which have dominant-negative or complementing activity. Mol Cell Biol. 1997; 17(4):2020-2029.
Xu B, Kim S, Kastan M B. Involvement of Brca1 in S-phase and G(2)-phase checkpoints after ionizing irradiation. Mol Cell Biol. 2001; 21(10):3445-3450.
Stagni V, Cirotti C, Barila D. Ataxia-Telangiectasia Mutated Kinase in the Control of Oxidative Stress, Mitochondria, and Autophagy in Cancer: A Maestro With a Large Orchestra. Front Oncol. 2018; 8:73.
Ditch S, Paull T T. The ATM protein kinase and cellular redox signaling: beyond the DNA damage response. Trends Biochem Sci. 2012; 37(1):15-22.
Zhou T, Chou J, Zhou Y, et al. Ataxia telangiectasia-mutated dependent DNA damage checkpoint functions regulate gene expression in human fibroblasts. Mol Cancer Res. 2007; 5(8):813-822.
Littlefield L G, Colyer S P, Joiner E E, DuFrain R J, Frome E, Cohen M M. Chromosomal radiation sensitivity in ataxia telangiectasia long-term lymphoblastoid cell lines. Cytogenet Cell Genet. 1981; 31(4):203-213.
Natarajan A T, Meyers M. Chromosomal radiosensitivity of ataxia telangiectasia cells at different cell cycle stages. Hum Genet. 1979; 52(1):127-132.
Keimling M, Volcic M, Csernok A, Wieland B, Dork T, Wiesmuller L. Functional characterization connects individual patient mutations in ataxia telangiectasia mutated (ATM) with dysfunction of specific DNA double-strand break-repair signaling pathways. FASEB J. 2011; 25(11):3849-3860.
Scott S P, Bendix R, Chen P, Clark R, Dork T, Lavin M F. Missense mutations but not allelic variants alter the function of ATM by dominant interference in patients with breast cancer. Proc Natl Acad Sci USA. 2002; 99(2):925-930.
Qi J, Shackelford R, Manuszak R, et al. Functional expression of ATM gene carried by HSV amplicon vector in vitro and in vivo. Gene Ther. 2004; 11(1):25-33.
Young D B, Jonnalagadda J, Gatei M, Jans D A, Meyn S, Khanna K K. Identification of domains of ataxia-telangiectasia mutated required for nuclear localization and chromatin association. J Biol Chem. 2005; 280(30):27587-27594.
Zhang N, Chen P, Khanna K K, et al. Isolation of full-length ATM cDNA and correction of the ataxia-telangiectasia cellular phenotype. Proc Natl Acad Sci USA. 1997; 94(15):8021-8026.
Ziv Y, Bar-Shira A, Pecker I, et al. Recombinant ATM protein complements the cellular A-T phenotype. Oncogene. 1997; 15(2):159-167.
Fernandes N, Sun Y, Chen S, et al. DNA damage-induced association of ATM with its target proteins requires a protein interaction domain in the N terminus of ATM. J Biol Chem. 2005; 280(15):15158-15164.
Chen S, Paul P, Price B D. ATM's leucine-rich domain and adjacent sequences are essential for ATM to regulate the DNA damage response. Oncogene. 2003; 22(41):6332-6339.
Sun Y, Xu Y, Roy K, Price B D. DNA damage-induced acetylation of lysine 3016 of ATM activates ATM kinase activity. Mol Cell Biol. 2007; 27(24):8502-8509.
Jiang X, Sun Y, Chen S, Roy K, Price B D. The FATC domains of PIKK proteins are functionally equivalent and participate in the Tip60-dependent activation of DNA-PKcs and ATM. J Biol Chem. 2006; 281(23):15741-15746.
Canman C E, Lim D S, Cimprich K A, et al. Activation of the ATM kinase by ionizing radiation and phosphorylation of p53. Science. 1998; 281(5383):1677-1679.
Mordes D A, Glick G G, Zhao R, Cortez D. TopBP1 activates ATR through ATRIP and a PIKK regulatory domain. Genes Dev. 2008; 22(11):1478-1489.
Baretic D, Pollard H K, Fisher D I, et al. Structures of closed and open conformations of dimeric human ATM. Sci Adv. 2017; 3(5):e1700933.
Lempiainen H, Halazonetis T D. Emerging common themes in regulation of PIKKs and PI3Ks. EMBO J. 2009; 28(20):3067-3073.
Perry J, Kleckner N. The ATRs, ATMs, and TORs are giant HEAT repeat proteins. Cell. 2003; 112(2):151-155.
Seidel J J, Anderson C M, Blackburn E H. A novel Tell/ATM N-terminal motif, TAN, is essential for telomere length maintenance and a DNA damage response. Mol Cell Biol. 2008; 28(18):5736-5746.
You Z, Chahwan C, Bailis J, Hunter T, Russell P. ATM activation and its recruitment to damaged DNA require binding to the C terminus of Nbs1. Mol Cell Biol. 2005; 25(13): 5363-5379.
Bakkenist C J, Kastan M B. DNA damage activates ATM through intermolecular autophosphorylation and dimer dissociation. Nature. 2003; 421(6922):499-506.
Kapoor N S, Curcio L D, Blakemore C A, et al. Multigene Panel Testing Detects Equal Rates of Pathogenic BRCA1/2 Mutations and has a Higher Diagnostic Yield Compared to Limited BRCA1/2 Analysis Alone in Patients at Risk for Hereditary Breast Cancer. Ann Surg Oncol. 2015; 22(10):3282-3288.
Tung N, Lin N U, Kidd J, et al. Frequency of Germline Mutations in 25 Cancer Susceptibility Genes in a Sequential Series of Patients With Breast Cancer. J Clin Oncol. 2016; 34(13):1460-1468.
Sun Y, Jiang X, Chen S, Fernandes N, Price B D. A role for the Tip60 histone acetyltransferase in the acetylation and activation of ATM. Proc Natl Acad Sci USA. 2005; 102(37): 13182-13187.
Decker B, Allen J, Luccarini C, et al. Rare, protein-truncating variants in ATM, CHEK2 and PALB2, but not XRCC2, are associated with increased breast cancer risks. J Med Genet. 2017; 54(11):732-741.
Sandoval N, Platzer M, Rosenthal A, et al. Characterization of ATM gene mutations in 66 ataxia telangiectasia families. Hum Mol Genet. 1999; 8(1):69-79.
Li A, Swift M. Mutations at the ataxia-telangiectasia locus and clinical phenotypes of A-T patients. Am J Med Genet. 2000; 92(3):170-177.
Mahon S M. Management of Individuals With a Mutation in the Ataxia Telangiectasia Mutated Gene. Oncol Nurs Forum. 2016; 43(1):114-117.
Williamson C T, Muzik H, Turhan A G, et al. ATM deficiency sensitizes mantle cell lymphoma cells to poly(ADP-ribose) polymerase-1 inhibitors. Mol Cancer Ther. 2010; 9(2):347-357.
Gilardini Montani M S, Prodosmo A, Stagni V, et al. ATM-depletion in breast cancer cells confers sensitivity to PARP inhibition. J Exp Clin Cancer Res. 2013; 32:95.
Stankovic T, Kidd A M, Sutcliffe A, et al. ATM mutations and phenotypes in ataxia-telangiectasia families in the British Isles: expression of mutant ATM and the risk of leukemia, lymphoma, and breast cancer. Am J Hum Genet. 1998; 62(2):334-345.
Taylor A M, Lam Z, Last J I, Byrd P J. Ataxia telangiectasia: more variation at clinical and cellular levels. Clin Genet. 2015; 87(3):199-208.
Hiel J A, van Engelen B G, Weemaes C M, et al. Distal spinal muscular atrophy as a major feature in adult-onset ataxia telangiectasia. Neurology. 2006; 67(2):346-349.
Guidugli L, Shimelis H, Masica D L, et al. Assessment of the Clinical Relevance of BRCA2 Missense Variants by Functional and Computational Approaches. Am J Hum Genet. 2018.
Shackelford R E, Innes C L, Sieber S O, Heinloth A N, Leadon S A, Paules R S. The Ataxia telangiectasia gene product is required for oxidative stress-induced G1 and G2 checkpoint function in human fibroblasts. J Biol Chem. 2001; 276(24):21951-21959.
Wood L D, Halvorsen T L, Dhar S, et al. Characterization of ataxia telangiectasia fibroblasts with extended life-span through telomerase expression. Oncogene. 2001; 20(3):278-288.
Tavtigian S V, Oefner P J, Babikyan D, et al. Rare, evolutionarily unlikely missense substitutions in ATM confer increased risk of breast cancer. Am J Hum Genet. 2009; 85(4):427-446.
Waddell N, Jonnalagadda J, Marsh A, et al. Characterization of the breast cancer associated ATM 7271T>G (V2424G) mutation by gene expression profiling. Genes Chromosomes Cancer. 2006; 45 (12): 1169-1181.
Reiman A, Srinivasan V, Barone G, et al. Lymphoid tumours and breast cancer in ataxia telangiectasia; substantial protective effect of residual ATM kinase activity against childhood tumours. Br J Cancer. 2011; 105(4):586-591.
Buzin C H, Gatti R A, Nguyen V Q, et al. Comprehensive scanning of the ATM gene with DOVAM-S. Hum Mutat. 2003; 21(2):123-131.
Matai M, Nahas S A, Du L T, et al. Functional and computational assessment of missense variants in the ataxia-telangiectasia mutated (ATM) gene: mutations with increased cancer risk. Hum Mutat. 2009; 30(1):12-21.
Andreassen P R, Skoufias D A, Margolis R L. Analysis of the spindle-assembly checkpoint in HeLa cells. Methods Mol Biol. 2004; 281:213-225.
Yamamoto K, Wang J, Sprinzen L, et al Kinase-dead ATM protein is highly oncogenic and can be preferentially targeted by Topo-isomerase I inhibitors. Elife. 2016; 5.
Park J Y, Toland A E, Olson S, et al. S64P and L104P variants of PALB2, identified in pancreatic cancer patients by targeted sequencing, compromise the DNA damage response. (In Preparation).
Andreassen P R, Toland A E. VUS and polygenic scores in risk-stratified screening and targeted therapies for breast and ovarian cancer. J Prec Med. 2018 (Invited Review—In Preparation).
Chenevix-Trench G, Spurdle A B, Gatei M, et al. Dominant negative ATM mutations in breast cancer families. J Natl Cancer Inst. 2002; 94(3):205-215.
Wilson K A, Stern D F. NFBD1/MDC1, 53BP1 and BRCA1 have both redundant and unique roles in the ATM pathway. Cell Cycle. 2008; 7(22):3584-3594.
Andreassen P R, D'Andrea A D, Taniguchi T. ATR couples FANCD2 monoubiquitination to the DNA-damage response. Genes Dev. 2004; 18(16):1958-1963.
Bick G, Zhang F, Meetei A R, Andreassen P R. Coordination of the recruitment of the FANCD2 and PALB2 Fanconi anemia proteins by an ubiquitin signaling network. Chromosoma. 2017; 126(3):417-430.
Chen C C, Kass E M, Yen W F, et al. ATM loss leads to synthetic lethality in BRCA1 BRCT mutant mice associated with exacerbated defects in homology-directed repair. Proc Natl Acad Sci USA. 2017; 114(29):7665-7670.
Kocher S, Rieckmann T, Rohaly G, et al. Radiation-induced double-strand breaks require ATM but not Artemis for homologous recombination during S-phase. Nucleic Acids Res. 2012; 40(17):8336-8347.
Bakr A, Oing C, Kocher S, et al. Involvement of ATM in homologous recombination after end resection and RAD51 nucleofilament formation. Nucleic Acids Res. 2015; 43 (6):3154-3166.
Taniguchi T, Garcia-Higuera I, Andreassen P R, Gregory R C, Grompe M, D'Andrea A D. S-phase-specific interaction of the Fanconi anemia protein, FANCD2, with BRCA1 and RAD51. Blood. 2002; 100(7):2414-2420.
Kass E M, Lim P X, Helgadottir H R, Moynahan M E, Jasin M. Robust homology-directed repair within mouse mammary tissue is not specifically affected by Brca2 mutation. Nat Commun. 2016; 7:13241.
Dang Y, Low W K, Xu J, et al. Inhibition of nonsense-mediated mRNA decay by the natural product pateamine A through eukaryotic initiation factor 4AIII J Biol Chem. 2009; 284(35):23613-23621. Houdayer C. In silico prediction of splice-affecting nucleotide variants. Methods Mol Biol. 2011; 760:269-281.
Swift M, Reitnauer P J, Morrell D, Chase C L. Breast and other cancers in families with ataxia-telangiectasia. N Engl J Med. 1987; 316(21):1289-1294.
Vorechovsky I, Luo L, Lindblom A, et al. ATM mutations in cancer families. Cancer Res. 1996; 56(18):4130-4133.
Gilad S, Chessa L, Khosravi R, et al. Genotype-phenotype relationships in ataxia-telangiectasia and variants. Am J Hum Genet. 1998; 62(3):551-561.
Starita L M, Islam M M, Banerjee T, et al. A Multiplex Homology-Directed DNA Repair Assay Reveals the Impact of More Than 1,000 BRCA1 Missense Substitution Variants on Protein Function. Am J Hum Genet. 2018.
Findlay G M, Daza R M, Martin B, et al. Accurate classification of BRCA1 variants with saturation genome editing. Nature. 2018.
Blomen V A, Majek P, Jae L T, et al. Gene essentiality and synthetic lethality in haploid human cells. Science. 2015; 350(6264):1092-1096.
Goldgar D E, Healey S, Dowty J G, et al. Rare variants in the ATM gene and risk of breast cancer. Breast Cancer Res. 2011; 13(4):R73.
Renault A L, Mebirouk N, Fuhrmann L, et al. Morphology and genomic hallmarks of breast tumours developed by ATM deleterious variant carriers. Breast Cancer Res. 2018; 20(1):28.
Abdulrahman A A, Heintzelman R C, Corbman M, Garcia F U. Invasive breast carcinomas with ATM gene variants of uncertain significance share distinct histopathologic features. Breast J. 2018; 24(3):291-297.
Brnich S E, Rivera-Munoz E A, Berg J S. Quantifying the potential of functional evidence to reclassify variants of uncertain significance in the categorical and Bayesian interpretation frameworks. Hum Mutat. 2018.
Spurdle A B, Whiley P J, Thompson B, et al. BRCA1 R1699Q variant displaying ambiguous functional abrogation confers intermediate breast and ovarian cancer risk. J Med Genet. 2012; 49(8):525-532.
Geoffroy-Perez B, Janin N, Ossian K, et al. Cancer risk in heterozygotes for ataxia-telangiectasia. Int. J Cancer. 2001; 93(2):288-293.
Andreassen P R, Lacroix F B, Lohez O D, Margolis R L. Neither p21WAF1 nor 14-3-3sigma prevents G2 progression to mitotic catastrophe in human colon carcinoma cells after DNA damage, but p21WAF1 induces stable G1 arrest in resulting tetraploid cells. Cancer Res. 2001; 61(20):7660-7668.
Andreassen P R, Lohez O D, Lacroix F B, Margolis R L. Tetraploid state induces p53-dependent arrest of nontransformed mammalian cells in G1. Mol Biol Cell. 2001; 12(5):1315-1328.
Andreassen P R, Margolis R L. Microtubule dependency of p34cdc2 inactivation and mitotic exit in mammalian cells. J Cell Biol. 1994; 127(3):789-802.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

1-26. (canceled)

27. A composition comprising a full-length BRCA2co gene or BRCA2co variant/mutation according to Table 2.

28. (canceled)

29. (canceled)

30. The composition of claim 27, said BRCA2co gene or BRCA2co variant/mutation being within a lentiviral plasmid, said lentiviral plasmid being within a cell according to Table 3.

31. The composition claim 27, wherein said BRCA2co variant/mutation comprises at least one non-endogenous restriction site.

32. The composition of claim 31, wherein said at least one non-endogenous restriction site is present at an interval of not more than 1500 base pair, or at an interval of between about 100 base pairs to about 1500 base pairs, or an interval of between 250 base pairs to 1000 base pairs, or about 500 base pairs to about 750 base pairs.

33. (canceled)

34. A composition comprising a lentiviral plasmid comprising an ATMco variant/mutation according to Table 4.

35. The composition of claim 34, wherein said lentiviral plasmid comprises a benign or pathogenic variant, a variant of uncertain significance, or a deletion mutation of ATMco.

36. The composition of claim 35, wherein said benign or pathogenic variant, a variant of uncertain significance, or a deletion mutation is selected from Table 2.

37. The composition of claim 34, wherein said ATMco variant/mutation comprises at least one non-endogenous restriction site.

38. The composition of claim 37, wherein said at least one non-endogenous restriction site is present at an interval of not more than 1500 base pair, or at an interval of between about 100 base pairs to about 1500 base pairs, or an interval of between 250 base pairs to 1000 base pairs, or about 500 base pairs to about 750 base pairs.

39. (canceled)

40. A composition comprising a lentiviral vector and a codon-optimized gene.

41. The composition of claim 40 wherein said codon-optimized gene is selected from ataxia telangiectasia mutated serine/threonine protein kinase (ATM); ataxia telangiectasia and Rad3-related protein kinase (ATR); breast cancer 1, early onset (BRCA1); breast cancer 2, early onset (BRCA2); checkpoint kinase 1 (CHEK1); Fanconi anemia complementation group M (FANCM); and protein kinase, DNA-activated, catalytic subunit (PRKDC).

42. The composition of claim 41, wherein said codon-optimized gene comprises at least one non-endogenous restriction site.

43. The composition of claim 42, wherein said at least one non-endogenous restriction site is present at an interval of not more than 1500 base pair, or at an interval of between about 100 base pairs to about 1500 base pairs, or an interval of between 250 base pairs to 1000 base pairs, or about 500 base pairs to about 750 base pairs.

44. The composition of claim 40, wherein said codon-optimized gene has a length of greater than about 6 kb.

45. The composition of claim 40, wherein said codon-optimized gene or variant can be expressed at a level at or above endogenous levels of a wild-type version of said codon-optimized gene.

46. The composition of claim 27, wherein said composition comprises a lentiviral plasmid.

47. The composition of claim 46 wherein said lentiviral plasmid comprises a benign or pathogenic variant, a variant of uncertain significance, or a deletion mutation of BRCA2co.


SEQUENCE LISTING (1 sequence)

SEQ ID NO: 1 - DNA, 19 bp, Artificial Sequence
  Target Sequence
gaagaatgcaggtttaata